(19) United States
(12) Patent Application Publication
(10) Pub. No.: US 2008/0249019 A1
(43) Pub. Date: Oct. 9, 2008

(54) TREATMENT OF MUCUS HYPERSECRETION

(75) Inventors: Keith Alan Foster, Salisbury (GB); John Chaddock, Salisbury (GB)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: SYNTAXIN, LTD., Abingdon (GB)

(21) Appl. No.: 12/101,749

(22) Filed: Apr. 11, 2008

Related U.S. Application Data
(63) Continuation-in-part of application No. 11/806,496, filed on May 31, 2007.
Application No. 11/806,496 is a continuation-in-part of application No. 11/518,213, filed on Sep. 11, 2006.
Application No. 11/518,213 is a continuation of application No. 10/633,698, filed on Aug. 5, 2003, now abandoned.
Application No. 10/633,698 is a continuation-in-part of application No. 09/763,669, filed on May 29, 2001, now Pat. No. 6,632,440.

(30) Foreign Application Priority Data
Aug. 25, 1998 (GB) 9818548.1

(51) Int. Cl. A61K 38/02; A61P 11/06; A61P 11/16

(52) U.S. Cl. 514/12

(57) ABSTRACT

A polypeptide and a nucleic acid encoding the polypeptide are described. The polypeptide includes a cytotoxic toxin, a targeting domain that selectively binds to a target cell that is a mucus-secreting cell and a translocating domain that translocates the cytotoxic toxin into the target cell. A nucleic acid encoding the polypeptide is also described. Also described is a pharmaceutical composition for topical administration to a patient suffering from mucus hypersecretion which includes the polypeptide and a formulation component selected from the group consisting of an excipient, an adjuvant and a propellant. Methods of treating hypersecretion of mucus, chronic obstructive pulmonary disease (COPD) or asthma are also described. These methods include administering to a patient in need thereof a therapeutically effective amount of the polypeptide.

This application is a continuation-in-part of U.S. patent application Ser. No. 11/806,496, filed May 31, 2007, which is a continuation-in-part of U.S. patent application Ser. No. 11/518,213, filed Sep. 11, 2006, which is a continuation of U.S. patent application Ser. No. 10/633,698, filed Aug. 5, 2003, which is a continuation-in-part of U.S. patent application Ser. No. 09/763,669, filed May 29, 2001, now U.S. Pat. No. 6,632,440, which is a national phase entry of PCT/GB99/02806, filed Aug. 25, 1999, which claims the benefit of GB 9818548.1, filed Aug. 25, 1998. Each of these applications is hereby incorporated by reference in their entirety.

Pursuant to the provisions of 37 C.F.R. §1.52(e)(5), the sequence listing text file named Sequence_Listing.txt, created on Apr. 11, 2008 and having a size of 93,637 bytes, and which is being submitted herewith, is incorporated by reference herein in its entirety.

### 1. FIELD OF THE INVENTION

The present invention relates to treatment of mucus hypersecretion, to compositions therefor and manufacture of those compositions. The present invention relates particularly, though not exclusively, to the treatment of chronic bronchitis in chronic obstructive pulmonary disease (COPD), asthma and other clinical conditions involving COPD.

### 2. DESCRIPTION OF RELATED ART

Mucus is a thin film of protective viscoelastic liquid which lines the airways. It is a 1-2% aqueous solution, in which the major components are the glycoconjugates known as mucins. Mucus, including the mucins, is secreted by mucus secretory cells, the surface epithelial goblet cells of the large airways and the mucus cells of the submucosal glands. Mucin release occurs by three mechanisms: constitutive secretion, regulated secretion and protease cell surface activity. Of these it is regulated secretion that responds to external stimuli and is amenable to therapeutic intervention in COPD and asthma. Regulated secretion involves release from intracellular granules by docking and fusion of the granules with the cell exterior to release their contents onto the airway surface. Fusion of the granules can either be with the plasma membrane of the epithelial cell or with the membrane of other granules leading to release via multigranular complexes fused at the cell surface. Regulated secretion of mucins is controlled by humoral factors and by neural mechanisms. The neural mechanisms in humans involve a minor contribution from the adrenergic, sympathetic pathway and a major cholinergic, parasympathetic component. Another important neural pathway regulating mucin secretion, particularly the hypersecretion of pathological conditions, is that of the Non-Adrenergic Non-Cholinergic (NANC) pathway. The NANC component involves both an orthodromic pathway involving neuropeptide and nonpeptide transmitters, and a local sensory efferent pathway involving antidromic fibres from sensory C fibres.

COPD is a common respiratory condition, being the fourth most common cause of death in middle age in the Western world. COPD comprises two related diseases, which usually occur together, emphysema and chronic bronchitis. The pathological basis of chronic bronchitis is mucus hypersecretion. The excessive, chronic bronchial secretion results in expectoration, and can last from a few days to many years. The mucus hypersecretion of COPD results in small airway obstruction producing reduced maximal respiratory flow and slow forced lung emptying. There is minimal reversal of the impaired airway function of COPD by bronchodilators and currently no effective therapy for the mucus hypersecretion.

Mucus hypersecretion is also a significant contributing factor to the pathophysiology of asthma. It is a key component in status asthmaticus, and contributes to the chronic symptoms and morbidity of asthma. The mucus hypersecretion component of asthma is not well controlled by current therapies, particularly in severe and chronic cases.

It would accordingly be desirable to treat, reduce or prevent the mucus hypersecretion that causes or leads to these disease conditions.

### SUMMARY OF THE INVENTION

Accordingly, the invention provides a method of treating mucus hypersecretion comprising inhibiting mucus secretion by mucus secreting cells and/or inhibiting neurotransmitter release from neuronal cells that control or direct mucus secretion. The invention further provides, in a second aspect, a compound, for use in the treatment of mucus hypersecretion, which inhibits mucus secretion by (i) inhibiting mucus secretion by mucus secreting cells, or (ii) inhibiting neurotransmitter release from neuronal cells controlling or directing mucus secretion.

In accordance with one aspect, the invention provides a polypeptide comprising:

(a) a cytotoxic toxin;

(b) a targeting domain that selectively binds to a target cell that is a mucus-secreting cell; and

(c) a translocating domain that translocates the cytotoxic toxin into the target cell.

In accordance with another aspect, the invention provides a nucleic acid encoding said polypeptide.

In accordance with another aspect, the invention provides a pharmaceutical composition for topical administration to a patient suffering from mucus hypersecretion, comprising (i) a polypeptide as defined above; and (ii) a formulation component selected from the group consisting of an excipient, an adjuvant and a propellant.

In another aspect, the invention provides a method of treating hypersecretion of mucus, comprising administering to a patient in need thereof, a therapeutically effective amount of said polypeptide; wherein following said administration, hypersecretion of mucus is reduced.

In another aspect, the invention provides a method of treating chronic obstructive pulmonary disease (COPD) or asthma, comprising administering to a patient in need thereof, a therapeutically effective amount of a polypeptide as defined above; wherein following said administration, hypersecretion of mucus is reduced.

An advantage of the invention is that an agent for effective treatment of mucus hypersecretion and associated disease states is now provided and used, offering a relief to sufferers where hitherto there was no such agent available.

The present invention thus represents a new different approach to treatment of mucus hypersecretion by inhibiting secretory processes, namely one or other or both of the mucus secretion by mucus secretory cells and the secretion of neurotransmitters regulating mucus secretion. Agents of the present invention reduce mucus secretion and/or prevent the hypersecretion of COPD and asthma, and any other disease in which mucus hypersecretion is a causative element.

### DETAILED DESCRIPTION OF THE INVENTION

In one embodiment, a compound of the invention typically inhibits exocytosis in mucus secreting cells or neurones that control or direct mucus secretion. This compound is administered to a patient suffering from mucus hypersecretion and inhibition of exocytosis in the cells specified results in reduction of secretion of mucus. Specific disease states caused by or exacerbated by hypersecretion are localised to the airways, and hence an embodiment of the invention comprises topical administration to the airways or to a selected region or to a selected portion of the airways of a compound that inhibits exocytosis in mucus secreting cells or in neurones that control or direct mucus secretion.

A compound of embodiments of the invention is a polypeptide that consists of or comprises an inhibiting domain which inhibits exocytosis in the mucus secreting cell or inhibits exocytosis in a neuronal cell, thereby directly inhibiting exocytosis of mucus or one or more mucus components or indirectly inhibiting mucus secretion by inhibiting exocytosis of neurotransmitter which would in turn lead to or otherwise stimulate mucus secretion. The inhibiting domain can suitably comprise a light chain of a clostridial neurotoxin, or a fragment or variant thereof which inhibits exocytosis. In an alternative embodiment, the inhibiting domain can be replaced with a cytotoxic toxin.

The compound preferably further comprises a translocating domain that translocates the inhibiting domain or cytotoxic toxin into the cell. This domain may comprise a H_{N} region of a botulinum polypeptide, or a fragment or variant thereof that translocates the inhibiting domain or cytotoxic toxin into the cell.

The compound preferably comprises a targeting domain which binds to (i) a mucus secreting cell, or (ii) a neuronal cell controlling or directing mucus secretion. The compound is thus rendered specific for these cell types. It is also optional for the compound to be relatively non-specific but for inhibition of mucus secretion to be achieved via targeting of the compound through choice of route of administration—the compound is hence preferably administered to mucus secreting epithelial cells in the airways, specifically in the lungs. Whilst a non-specific compound of the invention may affect exocytosis in many cells of a wide range of types, generally only those cells that are stimulated will be affected and these stimulated cells will in typical disease states be those that are secreting mucus and contributing to disease.

When present, suitable targeting domains include, but are not restricted to, a domain selected from Substance P, VIP, beta-2-adrenoreceptor agonists, gastrin releasing peptide and calcitonin gene related peptide. The precise cells targeted in preferred embodiments of the invention are selected from (a) cells that secrete mucins, such as epithelial goblet cells and submucosal gland mucus secreting cells, (b) cells that secrete aqueous components of mucus, such as Clara cells and serous cells, and (c) cells that control or direct mucus secretion, such as “sensory-efferent” C-fibres, or NANC neural system fibres. The compound may be administered as a substantially pure preparation all targeted to the same cell type, or may be a mixture of compounds targeted respectively to different cells.

The compound of specific embodiments of the invention comprises first, second and third domains. The first domain is adapted to cleave one or more vesicle or plasma-membrane associated proteins essential to exocytosis. This domain prevents exocytosis once delivered to a targeted cell. Alternatively, the first domain is adapted to be cytotoxic and induce cell death. The second domain translocates the compound into the cell. This domain delivers the first domain into the cell. The third domain binds to the target cell, ie binds to (i) a mucus secreting cell, or (ii) a neuronal cell controlling or directing mucus secretion, and may be referred to as a targeting moiety (“TM”). The compound may be derived from a toxin and it is preferred that such a compound is free of clostridial neurotoxin and free of any clostridial neurotoxin precursor that can be converted into toxin. Botulinum and tetanus toxin are suitable sources of domains for the compounds of the invention.

The compound of further specific embodiments of the invention comprises first, second and third domains. The first domain is a cytotoxic toxin, and induces cell death upon delivery to a target cell. The second domain translocates the compound into the cell. This domain delivers the first domain into the cell. The third domain binds to the target cell, ie binds to (i) a mucus secreting cell, or (ii) a neuronal cell controlling or directing mucus secretion, and may be referred to as a targeting moiety (“TM”).

In use, the agent of specific embodiments of the invention has a number of discrete functions. It binds to a surface structure (the Binding Site {BS}) which is characteristic of, and has a degree of specificity for, the relevant secretory cells and or neurones in the airways responsible for secretion of mucus and or regulation of said secretion. It enters the cell to which it binds by a process of endocytosis. Only certain cell surface BSs can undergo endocytosis, and preferably the BS to which the agent binds is one of these.

In one embodiment, the agent enters the cytosol, and modifies components of the exocytotic machinery present in the relevant secretory cells and or neurones in the airways responsible for secretion of mucus and or regulation of said secretion. In an alternative embodiment, the agent enters the cytosol and induces cell death of the secretory cells or neurones responsible for secretion of mucus or regulation of said secretion.

Surprisingly, agents of specific embodiments of the present invention for treatment of mucus hypersecretion can be produced by modifying a clostridial neurotoxin or fragment thereof. The clostridial neurotoxins share a common architecture of a catalytic L-chain (LC, ca 50 kDa) disulphide linked to a receptor binding and translocating H-chain (H_{C}, ca 100 kDa). The H_{C} polypeptide is considered to comprise all or part of two distinct functional domains. The carboxy-terminal half of the H_{C} (ca 50 kDa), termed the H_{C} domain, is involved in the high affinity, neurospecific binding of the neurotoxin to cell surface receptors on the target neuron, whilst the amino-terminal half, termed the H_{N} domain (ca 50 kDa), is considered to mediate the translocation of at least some portion of the neurotoxin across cellular membranes such that the functional activity of the LC is expressed within the target cell. The H_{N} domain also has the property, under conditions of low pH, of forming ion-permeable channels in lipid membranes, this may in some manner relate to its translocation function.

For botulinum neurotoxin type A (BoNT/A) these domains are considered to reside within amino acid residues 872-1296 for the H_{C}, amino acid residues 449-871 for the H_{N} and residues 1-448 for the LC. Digestion with trypsin effectively degrades the H_{C} domain of the BoNT/A to generate a non-toxic fragment designated LH_{N}, which is no longer able to bind to and enter neurons. The LH_{N} fragment so produced also has the property of enhanced solubility compared to both the parent holotoxin and the isolated LC.

It is therefore possible to provide functional definitions of the domains within the neurotoxin molecule, as follows:

(A) clostridial neurotoxin light chain: A metalloprotease exhibiting high substrate specificity for vesicle and/or plasma membrane associated proteins involved in the exocytotic process. In particular, it cleaves one or more of SNAP-25, VAMP (synaptobrevin/cellubrevin) and syntaxin.

(B) clostridial neurotoxin heavy chain H_{N} domain: A portion of the heavy chain which enables translocation of that portion of the neurotoxin molecule such that a functional expression of light chain activity occurs within a target cell.

The domain responsible for translocation of the endopeptidase activity, following binding of neurotoxin to its specific cell surface receptor via the binding domain, into the target cell.

The domain responsible for formation of ion-permeable pores in lipid membranes under conditions of low pH.

The domain responsible for increasing the solubility of the entire polypeptide compared to the solubility of light chain alone.

(C) clostridial neurotoxin heavy chain H_{C} domain. A portion of the heavy chain which is responsible for binding of the native holotoxin to cell surface receptor(s) involved in the intoxicating action of clostridial toxin prior to internalisation of the toxin into the cell.

The identity of the cellular recognition markers for these toxins is currently not understood and no specific receptor species have yet been identified although Kozaki et al. have reported that synaptotagmin may be the receptor for botulinum neurotoxin type B. It is probable that each of the neurotoxins has a different receptor.

By covalently linking a clostridial neurotoxin, or a hybrid of two clostridial neurotoxins, in which the H_{C} region of the H-chain has been removed or modified, to a new molecule or moiety, the Targeting Moiety (TM), that binds to a BS on the surface of the relevant secretory cells and or neurones in the airways responsible for secretion of mucus and or regulation of said secretion, a novel agent capable of inhibiting mucus secretion is produced. A further surprising aspect of the present invention is that if the L-chain of a clostridial neurotoxin, or a fragment of the L-chain containing the endopeptidase activity, is covalently linked to TM which can also effect internalisation of the L-chain, or a fragment of the L-chain containing the endopeptidase activity, into the cytoplasm of the relevant secretory cells and or neurones in the airways responsible for secretion of mucus and or regulation of said secretion, this also produces a novel agent capable of inhibiting mucus secretion.

Accordingly, the invention may thus provide a compound containing a first domain equivalent to a clostridial toxin light chain and a second domain providing the functional aspects of the H_{N} of a clostridial toxin heavy chain, whilst lacking the functional aspects of a clostridial toxin H_{C} domain, and a third domain which binds to the target mucus secreting or mucus secretion controlling cell.

In an alternative embodiment, a novel agent capable of inhibiting mucus secretion is produced from a clostridial neurotoxin, or a hybrid of two clostridial neurotoxins, in which the H_{C} region of the H-chain has been removed or modified, is covalently linked to a TM and is further modified by replacing the L-chain with a cytotoxic toxin, or fragment or variant thereof having cytotoxic activity.

In a further embodiment of the present invention, a cytotoxic toxin (or fragment or variant thereof having cytotoxic activity) is covalently linked to TM which can also effect internalisation of the cytotoxic toxin or fragment into the cytoplasm of the relevant secretory cells and or neurones in the airways responsible for secretion of mucus and or regulation of said secretion, thereby producing a novel agent capable of inhibiting mucus secretion.

Accordingly, the invention may provide a compound containing a first domain which is equivalent to a cytotoxic toxin and a second domain providing the functional aspects of the H_{N} of a clostridial toxin heavy chain, whilst lacking the functional aspects of a clostridial toxin H_{C} domain, and a third domain which binds to the target mucus secreting or mucus secretion controlling cell. This compound is effective in stopping mucus secretion by causing the death of the relevant secretory cells and or neurones in the airways responsible for secretion of mucus and or regulation of said secretion.

For the purposes of the invention, the functional property or properties of the H_{N} of a clostridial toxin heavy chain that are to be exhibited by the second domain of the polypeptide of the invention is translocation of the first domain into a target cell once the compound is proximal to the target cell. References hereafter to a H_{N} domain or to the functions of a H_{N} domain are references to this property or properties. The second domain is not required to exhibit other properties of the H_{N} domain of a clostridial toxin heavy chain. A second domain of the invention can thus be relatively insoluble but retain the translocation function of a native toxin. This is of use if solubility is not essential to its administration or if necessary solubility is imparted to a composition made up of that domain and one or more other components by one or more of said other components.

The translocating domain may be obtained from a microbial protein source, in particular from a bacterial or viral protein source. It is well documented that certain domains of bacterial toxin molecules are capable of forming such pores. It is also known that certain translocation domains of virally expressed membrane fusion proteins are capable of forming such pores. Such domains may be employed in the present invention.

Hence, in one embodiment, the translocating domain is a translocating domain of an enzyme, such as a bacterial or viral toxin. One such molecule is the heavy chain of a clostridial neurotoxin, for example botulinum neurotoxin type A. Other sources of bacterial toxin translocating domains include diphtheria toxin and domain II of pseudomonas exotoxin.

Other sources of translocating domains include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded “spike proteins” have particular application in the context of the present invention, for example, the E1 protein of SFV and the G protein of the G protein of VSV.

Preferably it has been found to use only those portions of the protein molecule capable of pore-formation within the endosomal membrane.

Methodology to enable assessment of membrane fusion and thus identification of translocation domains suitable for use in the present invention are provided by Methods in Enzymology Vol 220 and 221, Membrane Fusion Techniques, Parts A and B, Academic Press 1993.

Examples of preferred translocating domains for use in the present invention are listed in the table below. The below-listed citations are all herein incorporated by reference.

| Translocation domain | Amino acid | |
|---|---|---|
| source | residues | References |
| Diphtheria toxin | 194-380 | Silverman et al., 1994, J. |
| | | Biol. Chem. 269, 22524-22532 |
| | | London E., 1992, |
| | | Biochem. Biophys. Acta., 1113, 25-51 |
| Domain II of | 405-613 | Prior et al., 1992, |
| pseudomonas exotoxin | | Biochemistry 31, 3555-3559; |
| | | Kihara & Pastan, |
| | | 1994, Bioconj Chem. 5, 532-538 |
| Influenza virus | GLFGAIAGFIENGW | Plank et al., 1994, J. Biol. |
| haemagglutinin | EGMIDGWYG (SEQ | Chem. 269, 12918-12924; |
| | ID NO: 21), and | Wagner et al, 1992, PNAS, |
| | variants thereof | 89, 7934-7938 Murata et |
| | | al., 1992, Biochemistry 31, 1986-1992 |
| Semliki Forest virus | Translocation | Kielian et al., 1996, J Cell |
| fusogenic protein | domain | Biol. 134(4), 863-872 |
| Vesicular Stomatitis | 118-139 | Yao et al., 2003, Virology |
| virus glycoprotein G | | 310(2), 319-332 |
| SER virus F protein | Translocation | Seth et al., 2003, J Virol |
| | domain | 77(11) 6520-6527 |
| Foamy virus envelope | Translocation | Picard-Maureau et al., |
| glycoprotein | domain | 2003, J Virol. 77(8), 4722-4730. |

Use of the translocating domains listed in the above table includes use of sequence variants thereof. A variant may comprise one or more conservative nucleic acid substitutions and/or nucleic acid deletions or insertions, with the proviso that the variant possesses the requisite translocating function. A variant may also comprise one or more amino acid substitutions and/or amino acid deletions or insertions, so long as the variant possesses the requisite translocating function.

The only functional requirement of the translocating domain is that it is capable of forming appropriate pores in the endosomal membrane. A number of routine methods are available for confirming that a particular translocating domain has the requisite translocating activity, and thus to determine the presence of a translocating domain. Shone et al. (1987), and Blaustein et al. (1987) provide details of two very simple assays to confirm that any particular bacterial translocating domain has the requisite translocating activity. Shone (1987) describes a simple in vitro assay employing liposomes, which are challenged with a test molecule. The presence of a molecule having the requisite translocating function is confirmed by release from the liposomes of K+ and/or labelled AND. Blaustein (1987) describes a simple in vitro assay employing planar phospholipid bilayer membranes, which are challenged with a test molecule. The presence of a molecule having the requisite translocation function is confirmed by an increase in conductance across the phospholipid membrane. The polypeptide of the invention may be obtained by expression of a recombinant nucleic acid, preferably a DNA, and is a single polypeptide, that is to say not cleaved into separate light and heavy chain domains. The polypeptide is thus available in convenient and large quantities using recombinant techniques.

The cytotoxic toxin may be obtained from a microbial, animal or plant source.

Microbial sources of cytotoxic toxins include bacterial and fungal sources.

Bacterial sources of cytotoxic toxins include gram positive bacteria and gram negative bacteria.

Examples of suitable cytotoxic toxins from gram positive bacteria include Diphtheria toxin from Corynebacterium diphtheriae, Exoenzyme C3 from Clostridium botulinum, and EDIN from Staphylococcus aureus.

Examples of suitable cytotoxic toxins from gram negative bacteria include VacA cytotoxin from Helicobacter pylori, Exotoxin A (ETA) from Pseudomonas aeruginosa, Pertussis toxin (PT) and Adenylate cyclase toxin (CYA) from Bordetella pertussis, Heat labile enterotoxin (LT) and Shiga-like toxin A chain from Escherichia coli, Cholera toxin from Vibrio cholerae, and Shiga-toxin A chain from Shigella dysenteriae.

Suitable cytotoxic toxins from fungal sources include a-sarcin from Aspergillus giganteus.

Animal sources of cytotoxic toxins include reptile sources.

Examples of suitable cytotoxic toxins from reptile sources include Cytotoxins C1 and C2 from Naja oxiana, and Cytotoxic phospholipase A2 from the Russells viper.

Examples of suitable cytotoxic toxins from plant sources include IRIP type I ribosome inactivating protein, Iris agglutinin b and Iris agglutinin r from Iris hollandica; Cinnamomin type II ribosome inactivating protein from Cinnamomum camphora; Ricin A-chain from Ricinus communis; Abrin A-chain from Abrus precatorius; Pokeweed antiviral protein from Phytolacca americana, Mistletoe lectin A subunit from Viscum album, Saporin 6 from Saponaria officinalis, Wheatgerm agglutinin A chain from Triticum vulgaris, Dianthin 30 from Diathus caryophyllus, Gelonin from Gelonium multiflorum, and Trichosanthin from Trichosanthes kirilowii.

In one embodiment, the first domain optionally comprises a fragment or variant of a clostridial toxin light chain. The fragment is optionally an N-terminal, or C-terminal fragment of the light chain, or is an internal fragment, so long as it substantially retains the ability to cleave the vesicle or plasma-membrane associated protein essential to exocytosis. Domains necessary for the activity of the light chain of clostridial toxins are described in J. Biol. Chem., Vol. 267, No. 21, July 1992, pages 14721-14729. The variant has a different peptide sequence from the light chain or from the fragment, though it too is capable of cleaving the vesicle or plasma-membrane associated protein. It is conveniently obtained by insertion, deletion and/or substitution of a light chain or fragment thereof. In embodiments of the invention described below, a variant sequence comprises (i) an N-terminal extension to a clostridial toxin light chain or fragment (ii) a clostridial toxin light chain or fragment modified by alteration of at least one amino acid (iii) a C-terminal extension to a clostridial toxin light chain or fragment, or (iv) combinations of 2 or more of (i)-(iii).

In an embodiment of the invention described in an example below, the toxin light chain and the portion of the toxin heavy chain are of botulinum toxin type A. In a further embodiment of the invention described in an example below, the toxin light chain and the portion of the toxin heavy chain are of botulinum toxin type B. The polypeptide optionally comprises a light chain or fragment or variant of one toxin type and a heavy chain or fragment or variant of another toxin type.

In a polypeptide according to the invention said second domain preferably comprises a clostridial toxin heavy chain H_{N} portion or a fragment or variant of a clostridial toxin heavy chain H_{N} portion. The fragment is optionally an N-terminal or C-terminal or internal fragment, so long as it retains the function of the H_{N} domain. Teachings of regions within the H_{N} responsible for its function are provided for example in Biochemistry 1995, 34, pages 15175-15181 and Eur. J. Biochem, 1989, 185, pages 197-203. The variant has a different sequence from the H_{N} domain or fragment, though it too retains the function of the H_{N} domain. It is conveniently obtained by insertion, deletion and/or substitution of a H_{N} domain or fragment thereof. In embodiments of the invention, described below, it comprises (i) an N-terminal extension to a H_{N} domain or fragment, (ii) a C-terminal extension to a H_{N} domain or fragment, (iii) a modification to a H_{N} domain or fragment by alteration of at least one amino acid, or (iv) combinations of 2 or more of (i)-(iii). The clostridial toxin is preferably botulinum toxin or tetanus toxin.

These polypeptides of the invention thus typically contain two or more polypeptide first and second domain, linked by di-sulphide bridges into composite molecules, and further linked to a third domain.

The TM provides specificity for the BS on the relevant neuronal and or secretory cells responsible for secretion of mucus in the airways. The TM component of the agent can comprise one of many cell binding molecules, including, but not limited to, antibodies, monoclonal antibodies, antibody fragments (Fab, F(ab)′2, Fv, ScFv, etc.), lectins, hormones, cytokines, growth factors or peptides.

It is known in the art that the H_{C} portion of the neurotoxin molecule can be removed from the other portion of the H-chain, known as H_{N}, such that the H_{N} fragment remains disulphide linked to the L-chain of the neurotoxin providing a fragment known as LH_{N}. Thus, in one embodiment of the present invention the LH_{N} fragment of a clostridial neurotoxin is covalently linked, using linkages which may include one or more spacer regions, to a TM.

In an alternative embodiment, a cytotoxic toxin is covalently linked to the H_{N} fragment, and the cytotoxin-H_{N} conjugate is covalently linked, using linkages which may include one or more spacer regions, to a TM.

The H_{C} domain of a clostridial neurotoxin may be mutated or modified, eg. by chemical modification, to reduce or preferably incapacitate its ability to bind the neurotoxin to receptors at the neuromuscular junction. This modified clostridial neurotoxin is then covalently linked, using linkages which may include one or more spacer regions, to a TM. In one embodiment, this modified clostridial neurotoxin-TM conjugate can be further modified by linkage to a cytotoxic toxin (or fragment or variant thereof having cytotoxic activity).

In one embodiment, the heavy chain of a clostridial neurotoxin, in which the H_{C} domain is mutated or modified, eg by chemical modification, to reduce or preferably incapacitate its ability to bind the neurotoxin to receptors at the neuromuscular junction, may be combined with the L-chain of a different clostridial neurotoxin. This hybrid, modified clostridial neurotoxin is then covalently linked, using linkages which may include one or more spacer regions, to a TM.

In one embodiment, the heavy chain of a clostridial neurotoxin, in which the H_{C} domain is mutated or modified, eg by chemical modification, to reduce or preferably incapacitate its ability to bind the neurotoxin to receptors at the neuromuscular junction, may be combined with a cytotoxic toxin (or fragment or variant thereof having cytotoxic activity). The modified heavy chain-cytotoxin conjugate is then covalently linked, using linkages which may include one or more spacer regions, to a TM.

In another embodiment of the invention, the H_{N} domain of a clostridial neurotoxin is combined with the L-chain of a different clostridial neurotoxin. This hybrid LH_{N} is then covalently linked, using linkages which may include one or more spacer regions, to a TM. In a further embodiment of the invention, the light chain of a clostridial neurotoxin, or a fragment of the light chain containing the endopeptidase activity, is covalently linked, using linkages which may include one or more spacer regions, to a TM which can also effect the internalisation of the L-chain, or a fragment of the L-chain containing the endopeptidase activity, into the cytoplasm of the relevant secretory and/or neuronal cells in the airways responsible for secretion of mucus and or regulation of said secretion. In a further embodiment, the L-chain of the above described construct can be replaced with a cytotoxic toxin (or fragment or variant thereof having cytotoxic activity).

The agent is optionally expressed recombinantly as a fusion protein which includes an appropriate TM in addition to any desired spacer regions. The recombinantly expressed agent may be derived wholly from the gene encoding one serotype of neurotoxin or may be a chimaera derived from genes encoding one or more serotypes. In another embodiment of the invention, the required LH_{N}, which may be a hybrid of an L and H_{N} from different clostridial types, is expressed recombinantly as a fusion protein with the TM, and may include one or more spacer regions.

The light chain of a clostridial neurotoxin, or a fragment of the light chain containing the endopeptidase activity, may be expressed recombinantly as a fusion protein with a TM which can also effect the internalisation of the L-chain, or a fragment of the L-chain containing the endopeptidase activity, into the cytoplasm of the relevant secretory and or neuronal cells in the airways responsible for secretion of mucus and or regulation of said secretion. The expressed fusion protein may also include one or more spacer regions.

In a further embodiment of the invention, the agent is expressed recombinantly as a fusion protein comprising a cytotoxic toxin, a translocating domain such as H_{N} and a TM. In a further embodiment, the agent is expressed recombinantly as a fusion protein comprising a cytotoxic toxin and a TM which can also effect the internalisation of the cytotoxic toxin into the cytoplasm of the relevant secretory and or neuronal cells in the airways responsible for secretion of mucus and or regulation of said secretion. The expressed fusion protein may also include one or more spacer regions.

A neurotoxin fragment as described in the present invention can be prepared by methods well known in the protein art, including, but not limited to, proteolytic cleavage or by genetic engineering strategies. Said fragment is preferably a non-toxic fragment. The conjugation may be chemical in nature using chemical or covalent linkers. Conjugates according to the present invention may be prepared by conventional methods known in the art.

According to a preferred embodiment of the present invention, the TM is a growth factor, preferably an epidermal growth factor (EGF), vascular endothelial growth factor, platelet-derived growth factor, keratinocyte growth factor, hepatocyte growth factor, transforming growth factor alpha, transforming growth factor beta. Additional preferred TMs include atrial natriuretic peptide, vasoactive intestinal peptide and THALW(H)T.

According to another preferred embodiment of the present invention, the TM is a peptide or protein that binds to a serous cell. A preferred example of such a TM is an integrin-binding protein.

Integrins are obligate heterodimer transmembrane proteins containing two distinct chains α (alpha) and β (beta) subunits. In mammals, 19 alpha and 8 beta subunits have been characterised—see Humphries, M. J. (2000), Integrin structure. Biochem Soc Trans. 28: 311-339, which is herein incorporated by reference thereto. Integrin subunits span through the plasma membrane, and in general have very short cytoplasmic domains of about 40-70 amino acids. Outside the cell plasma membrane, the alpha and beta chains lie close together along a length of about 23 nm, the final 5 nm NH₂-termini of each chain forming a ligand-binding region to which an agent of the present invention binds.

Preferred integrin-binding proteins of the present invention comprise the amino sequence Arg-Gly-Asp (“RGD”), which binds to the above-described ligand-binding region—see Craig. D et al. (2004), Structural insights into how the MIDAS ion stabilizes integrin binding to an RGD peptide under force. Structure, vol. 12, pp 2049-2058, which is herein incorporated by reference thereto.

In one embodiment, the integrin-binding protein TMs of the present invention have an amino acid length of between 3 and 100, preferably between 3 and 50, more preferably between 5 and 25, and particularly preferably between 5 and 15 amino acid residues.

The TMs of the present invention may form linear or cyclic structures.

Preferred integrin-binding TMs of the present invention include actin, alpha-actinin, focal contact adhesion kinase, paxillin, talin, RACK1, collagen, laminin, fibrinogen, heparin, phytohaemagglutinin, fibronectin, vitronectin, VCAM-1, ICAM-1, ICAM-2 and serum protein. Many integrins recognise the triple Arg-Gly-Asp (RGD) peptide sequence (Ruoslahti, 1996). The RGD motif is found in over 100 proteins including fibronectin, tenascin, fibrinogen and vitronectin. The RGD-integrin interaction is exploited as a conserved mechanism of cell entry by many pathogens including coxsackievirus (Roivaninen et al., 1991) and adenovirus (Mathias et al., 1994).

Additionally preferred integrin-binding TMs of the present invention include proteins selected from the following sequences: Arg-Gly-Asp-Phe-Val; Arg-Gly-Asp-{D-Phe}-{N-methyl-Val}; RGDFV; RGDfNMeV; GGRGDMFGA; GGCRGDMFGCA; GRGDSP; GRGESP; PLAEIDGIEL and CPLAEIDGIELC. Reference to the above sequences embraces linear and cyclic forms, together with peptides exhibiting at least 80%, 85%, 90%, 95%, 98%, 99% sequence identity with said sequences. All of said TMs preferably retain the “RGD” tri-peptide sequence.

In a third aspect, the invention provides a composition for use in treating mucus hypersecretion, comprising:

a compound according to any of the second aspect of the invention; and

at least one of a pharmaceutically acceptable excipient, adjuvant and/or propellant,

wherein the composition is for administration to the airways of a patient.

Aerosol administration is a preferred route of administration, though the present invention encompasses also any administration that delivers the compound to epithelia in the airways. Nasal administration is optional though buccal is preferred. The compound may thus be formulated for oral administration via aerosol or nebuliser or as a dry powder for inhalation using conventional excipients, adjuvants and/or propellants. The invention therefore further provides a pharmaceutical composition comprising a compound of the invention and a pharmaceutically acceptable carrier.

In use the compound will generally be employed in a pharmaceutical composition in association with a human pharmaceutical carrier, diluent and/or excipient, although the exact form of the composition will depend on the mode of administration. The compound may, for example, be employed in the form of an aerosol or nebulisable solution.

In a specific embodiment of the invention, described in further detail below, a polypeptide according to the invention comprises Substance P, and an L chain and a heavy chain H_{N} region of botulinum toxin A. In use, this may be administered to a patient by aerosol. A solution of the polypeptide is prepared and converted into an aerosol using a nebuliser for inhalation into the lungs of nebulised particles of diameter 1-5 microns.

In another specific embodiment of the invention, a polypeptide according to the invention comprises Substance P, and a cytotoxic toxin and a heavy chain H_{N} region of botulinum toxin A. In use, this polypeptide may be administered to a patient by aerosol. A solution of the polypeptide is prepared and converted into an aerosol using a nebuliser for inhalation into the lungs of nebulised particles of diameter 1-5 microns.

The dosage ranges for administration of the compounds of the present invention are those to produce the desired therapeutic effect. It will be appreciated that the dosage range required depends on the precise nature of the conjugate, the route of administration, the nature of the formulation, the age of the patient, the nature, extent or severity of the patient's condition, contraindications, if any, and the judgement of the attending physician. Wide variations in the required dosage, however, are to be expected depending on the precise nature of the conjugate. Variations in these dosage levels can be adjusted using standard empirical routines for optimisation, as is well understood in the art.

Fluid unit dosage forms are prepared utilising the compound and a pyrogen-free sterile vehicle. The compound, depending on the vehicle and concentration used, can be either dissolved or suspended in the vehicle. In preparing solutions the compound can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers maybe sterilised by autoclaving. Advantageously additives such as buffering, solubilising, stabilising, preservative or bactericidal, suspending or emulsifying agents and/or local anaesthetic agents may be dissolved in the vehicle.

Dry powders which are dissolved or suspended in a suitable vehicle prior to use may be prepared by filling pre-sterilised drug substance and other ingredients into a sterile container using aseptic technique in a sterile area. Alternatively the drug and other ingredients may be dissolved into suitable containers using aseptic technique in a sterile area. The product is then freeze dried and the containers are sealed aseptically.

Compositions suitable for administration via the respiratory tract include aerosols, nebulisable solutions or microfine powders for insufflation. In the latter case, particle size of less than 50 microns, especially less than 10 microns, is preferred. Such compositions may be made up in a conventional manner and employed in conjunction with conventional administration devices.

In further aspects of the invention, there is provided use of a compound that inhibits exocytosis in mucus secreting cells or neurones that control or direct mucus secretion in manufacture of a medicament for treating mucus hypersecretion, asthma or COPD.

The invention yet further provides a method of manufacture of a pharmaceutical composition, comprising: obtaining a clostridial neurotoxin and modifying it so as to remove or disable its H_{C} portion; or obtaining a clostridial neurotoxin the H_{C} portion of which has been removed or disabled; linking the toxin with a targeting moiety that binds to (i) a mucus secreting cell, or (ii) a neuronal cell that controls or directs mucus secretion.

The invention still further provides a method of manufacture of a pharmaceutical composition, comprising obtaining a first component having the domains: an inhibiting domain which inhibits exocytosis in a mucus secreting cell or neuronal cell that controls or directs mucus secretion; a translocating domain which translocates the inhibiting domain into the cell; and linking the first component to a second component that binds to (i) a mucus secreting cell, or (ii) a neuronal cell that controls or directs mucus secretion.

The invention also provides an alternative method of manufacture of a pharmaceutical composition, comprising: obtaining a clostridial neurotoxin and modifying it so as to remove or disable its H_{C} portion; or obtaining a clostridial neurotoxin the H_{C} portion of which has been removed or disabled; replacing the L-chain domain with a cytotoxic toxin (or cytotoxic fragment or variant thereof); and linking the toxin with a targeting moiety that binds to (i) a mucus secreting cell, or (ii) a neuronal cell that controls or directs mucus secretion.

The invention also provides an alternative method of manufacture of a pharmaceutical composition, comprising: obtaining a heavy chain of a clostridial neurotoxin and modifying it so as to remove or disable its H_{C} portion; or obtaining a clostridial neurotoxin heavy chain the H_{C} portion of which has been removed or disabled; linking said modified heavy chain with a cytotoxic toxin (or cytotoxic fragment or variant thereof); and linking the heavy chain-cytotoxin conjugate with a targeting moiety that binds to (i) a mucus secreting cell, or (ii) a neuronal cell that controls or directs mucus secretion.

The invention still further provides a method of manufacture of a pharmaceutical composition, comprising obtaining a first component having the domains: a cytotoxic toxin which causes cell death of a mucus secreting cell or neuronal cell that controls or directs mucus secretion; a translocating domain which translocates the cytotoxic toxin into the cell; and linking the first component to a second component that binds to (i) a mucus secreting cell, or (ii) a neuronal cell that controls or directs mucus secretion.

The first and second components are preferably formulated in an orally administrable composition in combination with one or more or an excipient, an adjuvant and a propellant.

### BRIEF DESCRIPTION OF THE DRAWINGS

Specific embodiments of the invention are now illustrated in the following examples with reference to the accompanying drawings in which:

FIG. 1 illustrates the preparation of the substance P-LH_{N}/A conjugate of Example 1

FIG. 2 shows Western blot detection of conjugated substance P-LH_{N}/A

FIG. 3 shows SDS-PAGE analysis of a WGA-LH_{N}/A purification scheme

FIGS. 4-6 show inhibition of neurotransmitter release from cultured neuronal cells;

FIG. 7 shows WGA-LH_{N}/A inhibits release from, but does not have specificity for, eDRG neurons.

FIG. 8 shows purification of a LH_{N}/C-EGF fusion protein. Using the methodology outlined in Example 9, a LH_{N}/C-EGF fusion protein was purified from E. coli BL21 cells. Briefly, the soluble products obtained following cell disruption were applied to a nickel-charged affinity capture column. Bound proteins were eluted with 100 mM imidazole, treated with Factor Xa to activate the fusion protein and remove the maltose-binding protein (MBP) tag, then re-applied to a second nickel-charged affinity capture column. Samples from the purification procedure were assessed by SDS-PAGE. Lane 1 & 6: Molecular mass markers (kDa), lane 2: Clarified crude cell lysate, lane 3: First nickel chelating Sepharose column eluant, lane 4: Factor Xa digested protein, lane 5: Purified LH_{N}/C-EGF under non-reducing conditions, lane 7: Purified LH_{N}/C-EGF under reduced conditions.

FIG. 9 shows purification of a LH_{N}/B-EGF fusion protein. Using the methodology outlined in Example 10, a LH_{N}/B-EGF fusion protein was purified from E. coli BL21 cells. Briefly, the soluble products obtained following cell disruption were applied to a nickel-charged affinity capture column. Bound proteins were eluted with 100 mM imidazole, treated with Factor Xa and enterokinase to activate the fusion protein and remove the maltose-binding protein (MBP) tag, then re-applied to a second nickel-charged affinity capture column. Samples from the purification procedure were assessed by SDS-PAGE. The final purified material in the absence and presence of reducing agent is identified in the lanes marked [−] and [+] respectively.

FIG. 10 shows purification of a LH_{N}/C-RGD fusion protein. Using the methodology outlined in Example 11, a LH_{N}/C-RGD fusion protein was purified from E. coli BL21 cells. Briefly, the soluble products obtained following cell disruption were applied to a nickel-charged affinity capture column. Bound proteins were eluted with 100 mM imidazole, treated with Factor Xa to activate the fusion protein and remove the maltose-binding protein (MBP) tag, then re-applied to a second nickel-charged affinity capture column. Samples from the purification procedure were assessed by SDS-PAGE. The final purified material in the absence and presence of reducing agent is identified in the lanes marked [−] and [+] respectively.

FIG. 11 shows purification of a LH_{N}/C-cyclic RGD fusion protein. Using the methodology outlined in Example 12, a LH_{N}/C-cyclic RGD fusion protein was purified from E. coli BL21 cells. Briefly, the soluble products obtained following cell disruption were applied to a nickel-charged affinity capture column. Bound proteins were eluted with 100 mM imidazole, treated with Factor Xa to activate the fusion protein and remove the maltose-binding protein (MBP) tag, then re-applied to a second nickel-charged affinity capture column. Samples from the purification procedure were assessed by SDS-PAGE. The final purified material in the absence and presence of reducing agent is identified in the lanes marked [−] and [+] respectively.

FIG. 12 shows purification of a LC/C-RGD-H_{N}/C fusion protein. Using the methodology outlined in Example 13, a LC/C-RGD-H_{N}/C fusion protein was purified from E. coli BL21 cells. Briefly, the soluble products obtained following cell disruption were applied to a nickel-charged affinity capture column. Bound proteins were eluted with 100 mM imidazole, treated with Factor Xa to activate the fusion protein and remove the maltose-binding protein (MBP) tag, then re-applied to a second nickel-charged affinity capture column. Samples from the purification procedure were assessed by SDS-PAGE. The final purified material in the absence and presence of reducing agent is identified in the lanes marked [−] and [+] respectively.

FIG. 13 shows VAMP cleavage activity of LH_{N}/B-EGF. Using the methodology outlined in Example 14, BoNT/B (•), LH_{N}/B (▪) and LH_{N}/B-EGF (▴) were assayed for VAMP cleavage activity.

FIG. 14 shows effect of EGF-LH_{N}/C on EGF/TNF alpha-induced mucin secretion in A549 cells. A549 cells were treated with the fusion or vehicle or medium alone for 24 hr followed by a 48 hr stimulation of the cells with EGF/TNF alpha (Stim) in the presence of the construct. Vehicle treated cells received fresh vehicle plus medium only. Media was collected and assayed for mucin content using the ELLA method with a human mucin standard curve. All mucin levels are shown in ng/ml calculated from the human mucin standard curve within each ELLA plate. Each concentration was assessed in triplicate and the figure is representative of seven experiments.

N=7

C=control (cell culture medium only)

VC=vehicle control (50 mM Hepes, 200 mM NaCl-eluant solution for the EGF-LH_{N}/C)

***p<0.01 vs C,

### p<0.001 vs Stim.

FIG. 15 shows effect of EGF-LH_{N}/C on EGF/TNF alpha-induced mucin secretion in NCI-H292 cells. Each concentration was assessed in triplicate and the figure is representative of four experiments. NCI-H292 cells were treated with the fusion or vehicle or medium alone for 48 hr followed by a 24 hr stimulation of the cells with EGF/TNF alpha (Stim) in the presence of the fusion. Vehicle treated cells received fresh vehicle plus medium only. Media was collected and assayed for mucin content using the ELLA method with a human mucin standard curve. All mucin levels are shown in ng/ml calculated from the human mucin standard curve within each ELLA plate. Each concentration was assessed in triplicate and the figure is representative of at least three experiments.

N=3

C=control (cell culture medium only)

VC=vehicle control (50 mM Hepes, 200 mM NaCl-eluant solution for the EGF-LH_{N}/C)

** p<0.01 vs C, ## p<0.01 vs S

FIGS. 16 and 17 show effect of EGF-LH_{N}/C on viability of A549 cells and of H292 cells, respectively. Cells were pre-incubated with EGF-LH_{N}/C for 48 h, followed by 24 h stimulation with EGF/TNFα in the continued presence of EGF-LH_{N}/C and viability assessed by MTT assay. Data are mean cell viability as % control (bars=SEM) of 7 independent experiments. C=control. V=highest concentration of vehicle for EGF-LH_{N}/C. No significant differences were found.

FIG. 18 shows Syntaxin cleavage in NCI-H292 cells by EGF-LH_{N}/C. Syntaxin cleavage in NCI-H292 cells by recombinant EGF-LH_{N}/C. Protein from cells treated for three days with the construct was analysed by Western blot. The Western blot shows the dose-dependent appearance of the cleavage product of syntaxin due to increasing concentrations of EGF-LH_{N}/C. The blot was probed using a polyclonal antibody raised to the AVKY sequence at the BoNT/C cleavage site in syntaxin. LH_{N}/C alone or with EGF as not internalised in sufficient quantities to cause detectable cleavage of syntaxin. EGF alone did not cause cleavage of syntaxin.

FIG. 19 shows effect of EGF-LH_{N}/C in secreted and intracellular MUC5AC in A549 cells; and

FIG. 20 shows effects of EGF-LH_{N}/C on MUC5B content in treated cells. Band density on the nitrocellulose blots was detected using a densitometer and has been plotted in Optical density units×area. As the EGF-LH_{N}/C concentration increases the level of MUC5AC or MUC5B detected in the medium decreases and the intracellular, cell lysate levels increase. Data is representative of two experiments.

C=control (cell culture medium only)

S=EGF & TNF alpha (20 ng/ml & 25 ng/ml respectively)

Stimulation=EGF & TNF alpha (20 ng/ml & 25 ng/ml respectively)

FIG. 21 shows effect of LH_{N}/C on mucin secretion in A549 cells. A549 cells treated for 48 hr with 1 nM LH_{N}/C then stimulated for 24 hr with EGF/TNF alpha in the continued presence of LH_{N}/C showed no inhibition in mucin secretion as measured using the ELLA. Cells treated 24 hr with EGF-LH_{N}/C then stimulated for 48 hr with EGF/TNF alpha in the presence of EGF-LH_{N}/C showed inhibition of mucin secretion in the same experiment.

C=control (cell culture medium only)

VC=vehicle control (50 mM HEPES, 200 mM NaCl-eluant solution for the LH_{N}/C)

Data are Mean and SEM for 2-4 independent experiments.

***p<0.001 vs C; ##p<0.01 vs Stim.

FIG. 22 shows effect of LH_{N}/B-EGF on stimulated mucin secretion in A549 cells. Cells were treated with the fusion or vehicle or medium alone for 48 hour followed by a 24 hour stimulation of the cells with EGF/TNF alpha (Stimulus) in the presence of the construct. Vehicle treated cells received fresh vehicle plus medium only. Medium was collected and assayed for mucin content using the ELLA method with a human mucin standard curve. All mucin levels are shown in ng/ml calculated from the human mucin standard curve within each ELLA plate. Each concentration was assessed in triplicate. EGF-LH_{N}/B concentration in nM.

C=control (cell culture medium only)

Stim=EGF & TNF alpha (20 ng/ml &25 ng/ml respectively)

VC=vehicle control (50 mM Hepes, 200 mM NaCl-eluant solution for the EGF-LH_{N}/B)

FIG. 23 shows effect of H_{N}-RGD-LC/C on EGF/TNF alpha-induced mucin secretion in A549 cells. A549 Cells treated for 48 hour with varying concentrations of H_{N}-RGD-LC/C then stimulated for 24 hour with EGF/TNF alpha in the continued presence of H_{N}-RGD-LC/C showed a dose-dependent inhibition of mucin release as measured using the ELLA. Each concentration was assessed in triplicate and the figure is representative of two experiments.

C=control (cell culture medium only)

S=EGF & TNF alpha (20 ng/ml &25 ng/ml respectively)

Stimulation=EGF & TNF alpha (20 ng/ml &25 ng/ml respectively)

FIG. 24 shows effect of RGD-LH_{N}/C on EGF/TNF alpha-induced mucin secretion in A594 cells. Cells treated for 48 h with RGD-LH_{N}/C then stimulated for 24 h with EGF/TNFα in the continued presence of RGD-LH_{N}/C showed a dose-dependent inhibition of mucin release as measured using the ELLA. Each concentration was assessed in triplicate and the figure is representative of two experiments.

FIG. 25 shows effect of C-RGD-LH_{N}/C on EGF/TNF alpha-induced mucin secretion in A549 cells. Cells treated for 48 h with C-RGD-H_{N}-LC/C then stimulated for 24 h with EGF/TNFα in the continued presence of C-RGD-H_{N}-LC/C showed a dose-dependent inhibition of mucin release as measured using the ELLA. Each concentration was assessed in triplicate and the figure is representative of two experiments.

### EXAMPLES

### Example 1

### Method for the Preparation of Substance P-LH_{N}/A Conjugates

The lyophilised peptide was rehydrated in 0.1% trifluoroacetic acid (TFA) to a final concentration of 10 mM. Aliquots of this solution were stored at −20° C. until use. The LH_{N}/A was desalted into PBSE (PBS containing 1 mM EDTA). The resulting solution (3-5 mg/ml) was reacted with a three- or four-fold molar excess of SPDP by addition of a 10 mM stock solution of SPDP in DMSO. After 3 hours at room temperature the reaction was terminated by desalting over a PD-10 column into PBSE.

A portion of the derivatised LH_{N}/A was removed from the solution and reduced with DTT (5 mM, 30 min). This sample was analysed spectrophotometrically at 280 nm and 343 nm to determine the degree of derivatisation. The degree of derivatisation achieved was typically 2 mol/mol.

The bulk of the derivatised LH_{N}/A and the substance P peptide were mixed in proportions such that the peptide was in four-fold molar excess. The conjugation reaction was allowed to proceed for >16 hours at 4° C.

The product mixture was centrifuged to clear any precipitate that had developed. The supernatant was applied to a PD-10 column equilibrated in PBS and protein fractions were eluted by addition of PBS. Peptide and reaction by-products eluted after the main peak of protein and were discarded.

The conjugate mixture was concentrated to >1 mg/ml by centrifugation through concentrators (with 10000 molecular weight exclusion limit). The concentrated conjugate mixture was analysed by SDS-PAGE and Western blotting (probed with anti-substance P antibody) to confirm linkage of substance P peptide to LH_{N}/A.

The method described is for linkage of substance P peptide covalently to LH_{N}/A via a SPDP linker. A sulphydryl residue is incorporated into the C-terminus of the substance P residue, in this case by the addition of a Cys residue. Alternative linkers are available, including linkers utilising similar chemistry of derivatisation but generating non-reducible covalent bonds between the linked species.

The Substance P peptide sequence used in this particular example is RPKPQQFFGLMC (SEQ ID NO:22), though alternative sequences are also suitable, e.g. CRPKPQQFFGLM (SEQ ID NO:23), i.e. substance P with an N-terminal Cys.

The method described does not make use of any tagging system (e.g. poly His) to purify the conjugate from free LH_{N}/A. This has been demonstrated to be a successful method for the preparation of opioid peptide LH_{N}/A such the receptor binding function of the peptide was not compromised. A similar approach can be applied to the synthesis of subP-LH_{N}/A.

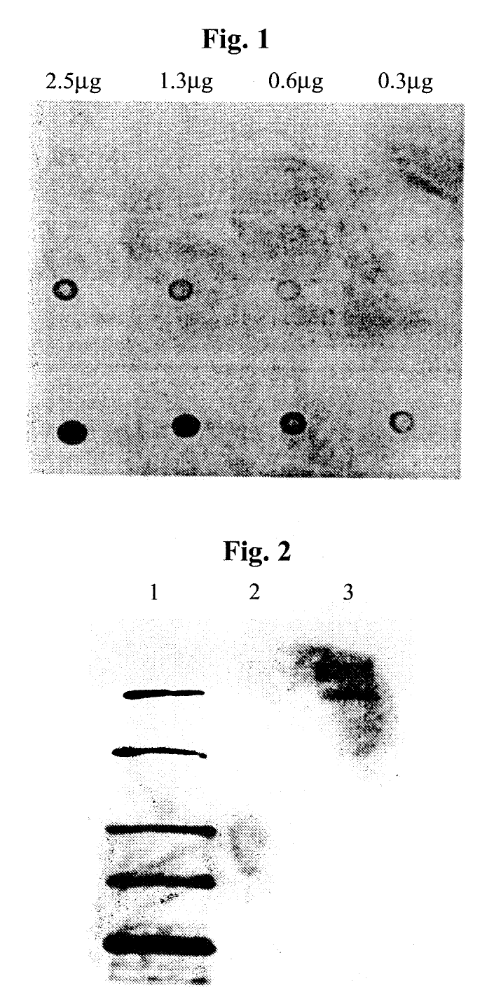

FIG. 1 illustrates the preparation of the substance P-LH_{N}/A conjugate of Example 1. In the results shown in FIG. 1, LH_{N}/A and substance P-LH_{N}/A samples at the concentrations indicated were applied to nitrocellulose and probed with rabbit anti-substance P antibody (upper two rows). The emergence of cross-reaction with the conjugate (second row), rather than the LH_{N}/A (first row), is indicative of substance P conjugated to LH_{N}/A. The lower control row illustrates the presence of LH_{N}/A.

FIG. 2 shows Western blot detection of conjugated substance P-LH_{N}/A. Samples of substance P-LH_{N}/A (lane 3) and LH_{N}/A (lane 2) were electrophoresed alongside molecular weight markers (lane 1). Detection of substance P by rat anti-substance P antisera indicated protein of approx. 100 kDa molecular weight in the conjugate lane, but no such band in the LH_{N}/A only lane. Thus the conjugated LH_{N}/A does contain substance P.

### Example 2

### Method for the Preparation of a Broad Specificity Agent

Conjugation and purification of WGA-LH_{N}/A. WGA (10 mg/ml in phosphate-buffered saline (PBS)) was reacted with an equal concentration of SPDP (10 mM in dimethyl sulphoxide (DMSO)) for one hour at ambient temperature. Reaction by-products were removed by desalting into PBS prior to reduction of the cross-linker with dithiothreitol. The thiopyridone and DTT were then removed by desalting into PBS to result in derivatised WGA (dWGA) with 1 mole -SH incorporated per mole of WGA.

LH_{N}/A at a concentration of 3-5 mg/ml in PBSE (PBS containing 1 mM EDTA) was reacted with a three or four-fold molar excess of SPDP (10 mM in DMSO). After 3 h at ambient temperature the reaction was terminated by desalting over a PD-10 column into PBSE.

The derivatised WGA (dWGA) and the derivatised LH_{N}/A (dLH_{N}/A) were mixed in a 3:1 molar ratio. After 16 h at 4° C. the mixture was centrifuged to clear any precipitate that had developed. The supernatant was concentrated by ultrafiltration before application to a Superose™ 12 column on an FPLC® chromatography system (Phamacia). The column was eluted with PBS and the fractions containing high molecular weight conjugate material (separated from free dWGA) were pooled and applied to PBS-washed N-acetylglucosamine-agarose (GlcNAc-agarose). WGA-LH_{N}/A conjugate bound to the GlcNAc-agarose and was eluted from the column by the addition of 0.3 M N-acetylglucosamine in PBS. The elution profile was followed at 280 nm and fractions containing conjugate were pooled, dialysed against PBS, and stored at 4° C. until use.

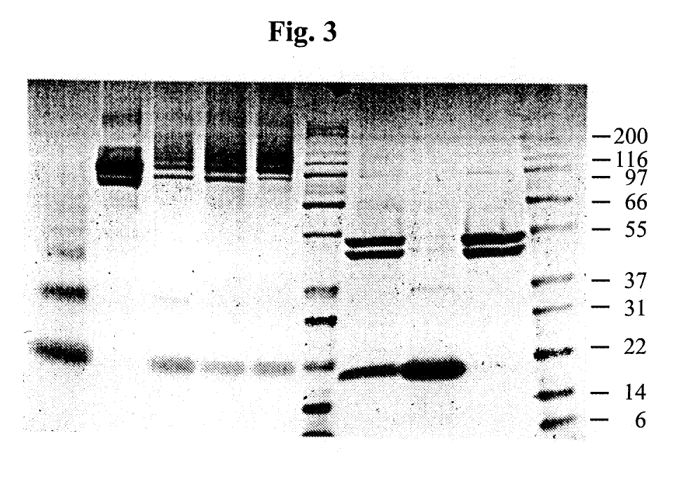

FIG. 3 shows SDS-PAGE analysis of WGA-LH_{N}/A purification scheme. Protein fractions were subjected to 4-20% polyacrylamide SDS-PAGE prior to staining with Coomassie blue. Lanes 6-8 were run in the presence of 0.1 M DTT. Lanes 1 (&7) and 2 (& 8) represent derivatised WGA and derivatised LH_{N}/A respectively. Lanes 3-5 represent conjugation mixture, post-Superose-12 chromatography and post GlcNAc-affinity chromatography respectively. Lanes 6 represents a sample of reduced final material. Approximate molecular masses (kDa) are indicated on the Figure.

### Example 3

### Preparation and Maintenance of Neuronal Cultures and Inhibition of Neurotransmitter Release

PC12 cells were seeded at a density of 4×10⁵ cells/well onto 24 well (matrigel coated) plates (NUNC™) from stocks grown in suspension. The cells were cultured for 1 week prior to use in RPMI, 10% horse serum, 5% foetal bovine serum, 1% L-glutamine. SH-SY5Y cells were seeded at a density of 5×10⁵ cells/well onto 24 well plates (FALCON™). The cells were cultured in HAM-F12:MEM (1:1 v/v), 15% foetal bovine serum, 1% MEM non-essential amino acids, 2 mM L-glutamine for 1 week prior to use. Embryonic spinal cord (eSC) neurons were prepared from spinal cords dissected from 14-15 day old foetal Sprague Dawley rats and were used after 21 days in culture using a modification of previously described method.

Inhibition of transmitter release. PC 2 cells or SH-SY5Y cells were washed with a balanced salt solution (BSS:137 mM NaCl, 5 mM KCl, 2 mM CaCl₂, 4.2 mM NaHCO₃, 1.2 mM MgCl₂, 0.44 mM KH₂PO₄, 5 mM glucose, 20 mM HEPES, pH7.4) and loaded for 1 hour with [³H]-noradrenaline (2 μCi/ml, 0.5 ml/well) in BSS containing 0.2 mM ascorbic acid and 0.2 mM pargyline. Cells were washed 4 times (at 15 minutes intervals for 1 hour) then basal release determined by a 5 minute incubation with BSS (5 mM K⁺). Cells were then depolarised with 100 mM K⁺ (BSS with Na⁺ reduced accordingly) for 5 minutes to determine stimulated release. Superfusate (0.5 ml) was removed to tubes on ice and briefly centrifuged to pellet any detached cells. Adherent cells were solubilised in 2 M acetic acid/0.1% trifluoroacetic acid (250 .mu.l/well). The quantity of released and non-released radio label was determined by liquid scintillation counting of cleared superfusates and cell lysates respectively. Total uptake was calculated by addition of released and retained radioactivity and the percentage release calculated ((released counts/total uptake counts)×100).

eSC neurons were loaded with [³H]-glycine for 30 minutes prior to determination of basal and potassium-stimulated release of transmitter. A sample of 0.2 M NaOH-lysed cells was used to determine total counts, from which % release could be calculated.

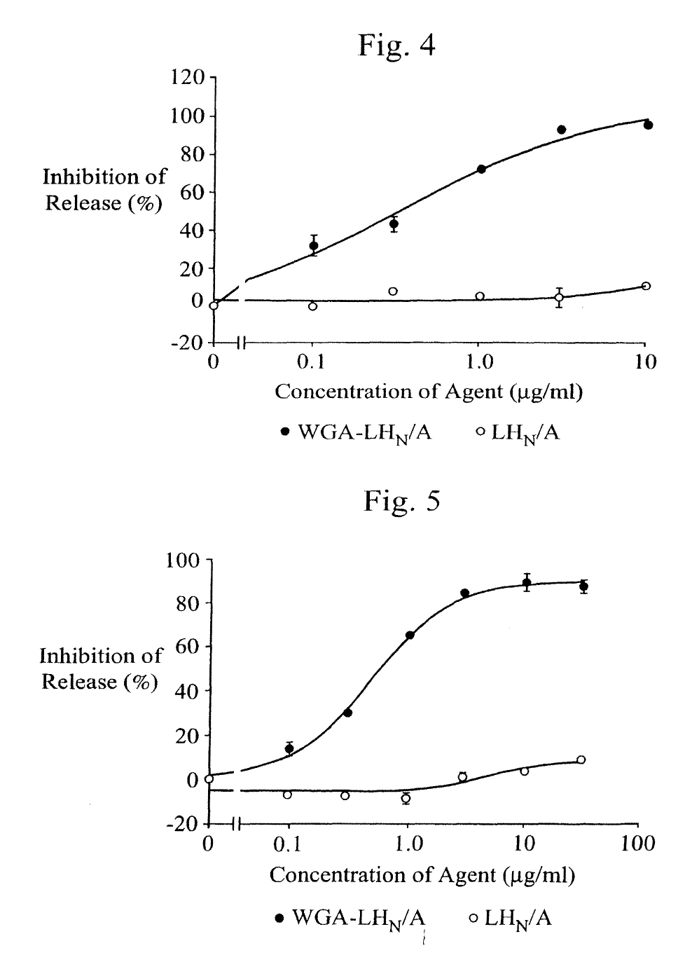

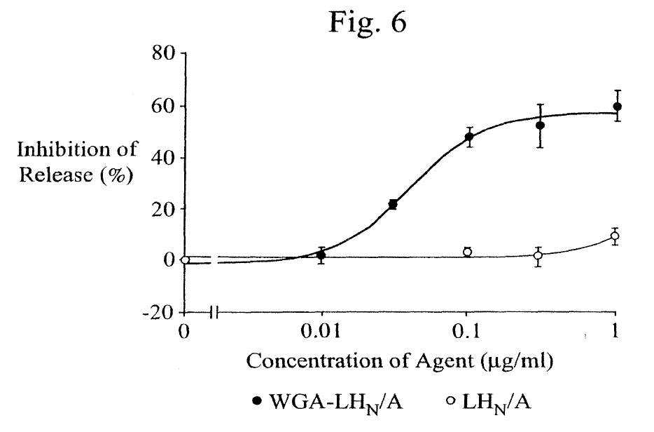

FIGS. 4-6 show inhibition of neurotransmitter release from cultured neuronal cells. PC12 (FIG. 4), SH-SY5Y cells (FIG. 5) and eSC neurons (FIG. 6) exposed for three days to a range of concentrations of WGA-LH_{N}/A (filled symbols) and LH_{N}/A (open symbols) were assessed for stimulated [³H]-noradrenaline release (SH-SY5Y and PC12 cells) or [³H]-glycine release (eSC) capability. Results are expressed as percentage inhibition compared to untreated controls. Each concentration was assessed in triplicate. For each cell type the dose response curve is representative of at least three experiments. Each point shown is the mean of at least three determinations +/−SE of the mean.

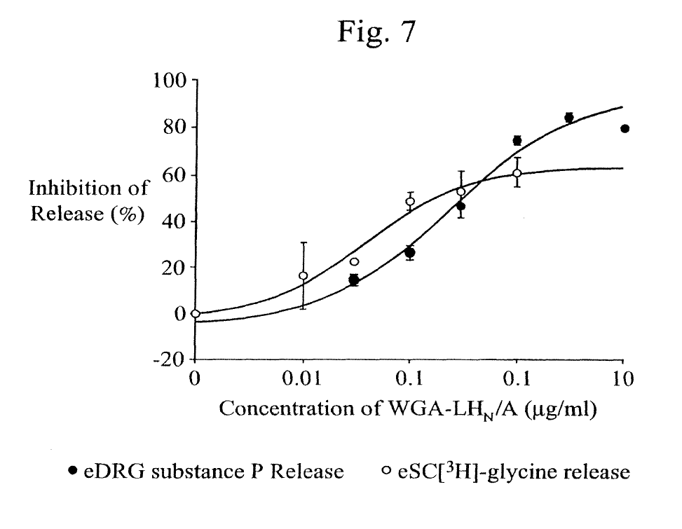

FIG. 7 shows dose-response curves of WGA-LH_{N}/A inhibition of eDRG substance P and eSC [³H]-glycine release. Cells were exposed to conjugate for three days. Representative curves are shown. Mean IC₅₀eDRG:0.32+0.05 μg/ml (n=4), eSC:0.06+/−0.01 μg/ml (n=3).

### Example 4

### Method for the Preparation of LC/B-Epidermal Growth Factor with a Translocation Domain from Diphtheria Toxin by Recombinant Expression

Using standard DNA manipulation procedures, the DNA encoding LC/B, diphtheria toxin amino acids 194-380, and epidermal growth factor are assembled in frame and inserted into an appropriate expression vector. Inserted between the LC/B DNA and the diphtheria toxin translocation domain is DNA encoding a short spacer sequence with a specific cleavable peptide bond (↓), bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form LC/B-DT.sub.194-380-EGF is achieved in E. coli using standard techniques. The expressed fusion protein is isolated from E. coli by standard purification techniques and cleaved by the specific activation enzyme prior to assessment in an in vitro cell model.

### Example 5

### Method for the Preparation of LC/C-Epidermal Growth Factor with a Translocation Domain from Pseudomonas Exotoxin by Recombinant Expression

Using standard DNA manipulation procedures, the DNA encoding LC/C, pseudomonas exotoxin amino acids 405-613, and epidermal growth factor are assembled in frame and inserted into an appropriate expression vector. Inserted between the LC/C DNA and the pseudomonas exotoxin translocation domain is DNA encoding a short spacer sequence with a specific cleavable peptide bond, bounded by a pair of cysteine amino acids. Examples of specific enzymes that may be used to activate the fusion protein include factor Xa (IEGR↓), (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form LC/C-PE₄₀₅₋₆₁₃-EGF is achieved in E. coli using standard techniques. The expressed fusion protein is isolated from E. coli by standard purification techniques and cleaved by the specific activation enzyme prior to assessment in an in vitro cell model.

### Example 6

### Method for the Preparation of LC/A-Epidermal Growth Factor with a Translocation Domain from Influenza Virus Haemagglutinin by Recombinant Expression

Using standard DNA manipulation procedures, the DNA encoding LC/A, GLFGAIAGFIENGWEGMIDGWYG (SEQ ID NO:21) from influenza virus haemagglutinin (HA), and epidermal growth factor are assembled in frame and inserted into an appropriate expression vector. Inserted between the LC/A DNA and the haemagglutinin sequence is DNA encoding a short spacer sequence with a specific cleavable peptide bond, bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form LC/A-HA-EGF is achieved in E. coli using standard techniques. The expressed fusion protein is isolated from E. coli by standard purification techniques and cleaved by the specific activation enzyme prior to assessment in an in vitro cell model.

The agent described in this invention can be used in vivo, either directly or as a pharmaceutically acceptable salt, for the treatment of conditions involving mucus hypersecretion, including COPD and asthma.

### Example 7

### Preparation of a LH_{N}/B Backbone Construct

The following procedure creates a clone for use as an expression backbone for multidomain fusion expression. This example is based on preparation of a serotype B based clone (SEQ ID1).

### Preparation of Cloning and Expression Vectors

pCR 4 (Invitrogen) is the chosen standard cloning vector chosen due to the lack of restriction sequences within the vector and adjacent sequencing primer sites for easy construct confirmation. The expression vector is based on the pMAL (NEB) expression vector which has the desired restriction sequences within the multiple cloning site in the correct orientation for construct insertion (BamHI-SalI-PstI-HindIII). A fragment of the expression vector has been removed to create a non-mobilisable plasmid and a variety of different fusion tags have been inserted to increase purification options.

### Preparation of LC/B

The LC/B is created by one of two ways:

The DNA sequence is designed by back translation of the LC/B amino acid sequence (obtained from freely available database sources such as GenBank (accession number P10844) or Swissprot (accession locus BXB_CLOBO) using one of a variety of reverse translation software tools (for example EditSeq best E. coli reverse translation (DNASTAR Inc.), or Backtranslation tool v2.0 (Entelechon)). BamHI/SalI recognition sequences are incorporated at the 5′ and 3′ ends respectively of the sequence maintaining the correct reading frame. The DNA sequence is screened (using software such as MapDraw, DNASTAR Inc.) for restriction enzyme cleavage sequences incorporated during the back translation. Any cleavage sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence containing the LC/B open reading frame (ORF) is then commercially synthesized (for example by Entelechon, Geneart or Sigma-Genosys) and is provided in the pCR 4 vector.

The alternative method is to use PCR amplification from an existing DNA sequence with BamHI and SalI restriction enzyme sequences incorporated into the 5′ and 3′ PCR primers respectively. Complementary oligonucleotide primers are chemically synthesised by a Supplier (for example MWG or Sigma-Genosys) so that each pair has the ability to hybridize to the opposite strands (3′ ends pointing “towards” each other) flanking the stretch of Clostridium target DNA, one oligonucleotide for each of the two DNA strands. To generate a PCR product the pair of short oligonucleotide primers specific for the Clostridium DNA sequence are mixed with the Clostridium DNA template and other reaction components and placed in a machine (the ‘PCR machine’) that can change the incubation temperature of the reaction tube automatically, cycling between approximately 94° C. (for denaturation), 55° C. (for oligonucleotide annealing), and 72° C. (for synthesis). Other reagents required for amplification of a PCR product include a DNA polymerase (such as Taq or Pfu polymerase), each of the four nucleotide dNTP building blocks of DNA in equimolar amounts (50-200 μM) and a buffer appropriate for the enzyme optimised for Mg2+ concentration (0.5-5 mM).

The amplification product is cloned into pCR 4 using either, TOPO TA cloning for Taq PCR products or Zero Blunt TOPO cloning for Pfu PCR products (both kits commercially available from Invitrogen). The resultant clone is checked by sequencing. Any additional restriction sequences which are not compatible with the cloning system are then removed using site directed mutagenesis (for example using Quickchange (Stratagene Inc.)).

### Preparation of H_{N}/B Insert

The H_{N} is created by one of two ways:

The DNA sequence is designed by back translation of the H_{N}/B amino acid sequence (obtained from freely available database sources such as GenBank (accession number P10844) or Swissprot (accession locus BXB_CLOBO)) using one of a variety of reverse translation software tools (for example EditSeq best E. coli reverse translation (DNASTAR Inc.), or Back translation tool v2.0 (Entelechon)). A PstI restriction sequence added to the N-terminus and XbaI-stop codon-HindIII to the C-terminus ensuring the correct reading frame in maintained. The DNA sequence is screened (using software such as MapDraw, DNASTAR Inc.) for restriction enzyme cleavage sequences incorporated during the back translation. Any sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector.

The alternative method is to use PCR amplification from an existing DNA sequence with PstI and XbaI-stop codon-HindIII restriction enzyme sequences incorporated into the 5′ and 3′ PCR primers respectively. The PCR amplification is performed as described above. The PCR product is inserted into pCR 4 vector and checked by sequencing. Any additional restriction sequences which are not compatible with the cloning system are then removed using site directed mutagenesis (for example using Quickchange (Stratagene Inc.)).

### Preparation of the Spacer (LC-H_{N} Linker)

The LC-H_{N} linker can be designed from first principle, using the existing sequence information for the linker as the template. For example, the serotype B linker (in this case defined as the inter-domain polypeptide region that exists between the cysteines of the disulphide bridge between LC and H_{N}) has the sequence KSVKAPG. This sequence information is freely available from available database sources such as GenBank (accession number P10844) or Swissprot (accession locus BXB_CLOBO). For generation of a specific protease cleavage site, the recognition sequence for enterokinase is inserted into the activation loop to generate the sequence VDEEKLYDDDDKDRWGSSLQ. Using one of a variety of reverse translation software tools (for example EditSeq best E. coli reverse translation (DNASTAR Inc.), or Backtranslation tool v2.0 (Entelechon)), the DNA sequence encoding the linker region is determined. BamHI/SalI and PstI/XbaI/stop codon/HindIII restriction enzyme sequences are incorporated at either end, in the correct reading frames. The DNA sequence is screened (using software such as MapDraw, DNASTAR Inc.) for restriction enzyme cleavage sequences incorporated during the back translation. Any sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector. If it is desired to clone the linker out of pCR 4 vector, the vector (encoding the linker) is cleaved with either BamHI+SalI or PstI+XbaI combination restriction enzymes. This cleaved vector then serves as the recipient vector for insertion and ligation of either the LC DNA (cleaved with BamHI/SalI) or H_{N} DNA (cleaved with PstI/XbaI). Once the LC or the H_{N} encoding DNA is inserted upstream or downstream of the linker DNA, the entire LC-linker or linker-H_{N} DNA fragment can the be isolated and transferred to the backbone clone.

As an alternative to independent gene synthesis of the linker, the linker-encoding DNA can be included during the synthesis or PCR amplification of either the LC or H_{N}.

### Assembly and Confirmation of the Backbone Clone

The LC or the LC-linker is cut out from the pCR 4 cloning vector using BamHI/SalI or BamHI/PstI restriction enzymes digests. The pMAL expression vector is digested with the same enzymes but is also treated with calf intestinal protease (CIP) as an extra precaution to prevent re-circularisation. Both the LC or LC-linker region and the pMAL vector backbone are gel purified. The purified insert and vector backbone are ligated together using T4 DNA ligase and the product is transformed with TOP10 cells which are then screened for LC insertion using BamHI/SalI or BamHI/PstI restriction digestion. The process is then repeated for the H_{N} or linker-H_{N} insertion into the PstI/HindIII or SalI/HindIII sequences of the pMAL-LC construct.

Screening with restriction enzymes is sufficient to ensure the final backbone is correct as all components are already sequenced confirmed, either during synthesis or following PCR amplification. However, during the sub-cloning of some components into the backbone, where similar size fragments are being removed and inserted, sequencing of a small region to confirm correct insertion is required.

### Example 8

### Preparation of a LH_{N}/C Backbone Construct

The following procedure creates a clone for use as an expression backbone for multidomain fusion expression. This example is based on preparation of a serotype C based clone (SEQ ID2).

### Preparation of Cloning and Expression Vectors

pCR 4 (Invitrogen) is the chosen standard cloning vector chosen due to the lack of restriction sequences within the vector and adjacent sequencing primer sites for easy construct confirmation. The expression vector is based on the pMAL (NEB) expression vector which has the desired restriction sequences within the multiple cloning site in the correct orientation for construct insertion (BamHI-SalI-PstI-HindIII). A fragment of the expression vector has been removed to create a non-mobilisable plasmid and a variety of different fusion tags have been inserted to increase purification options.

### Preparation of LC/C

The LC/C is created by one of two ways:

The DNA sequence is designed by back translation of the LC/C amino acid sequence (obtained from freely available database sources such as GenBank (accession number P18640) or Swissprot (accession locus BXC1_CLOBO) using one of a variety of reverse translation software tools (for example EditSeq best E. coli reverse translation (DNASTAR Inc.), or Backtranslation tool v2.0 (Entelechon)). BamHI/SalI recognition sequences are incorporated at the 5′ and 3′ ends respectively of the sequence maintaining the correct reading frame. The DNA sequence is screened (using software such as MapDraw, DNASTAR Inc.) for restriction enzyme cleavage sequences incorporated during the back translation. Any cleavage sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence containing the LC/C open reading frame (ORF) is then commercially synthesized (for example by Entelechon, Geneart or Sigma-Genosys) and is provided in the pCR 4 vector.

The alternative method is to use PCR amplification from an existing DNA sequence with BamHI and SalI restriction enzyme sequences incorporated into the 5′ and 3′ PCR primers respectively. Complementary oligonucleotide primers are chemically synthesised by a Supplier (for example MWG or Sigma-Genosys) so that each pair has the ability to hybridize to the opposite strands (3′ ends pointing “towards” each other) flanking the stretch of Clostridium target DNA, one oligonucleotide for each of the two DNA strands. To generate a PCR product the pair of short oligonucleotide primers specific for the Clostridium DNA sequence are mixed with the Clostridium DNA template and other reaction components and placed in a machine (the ‘PCR machine’) that can change the incubation temperature of the reaction tube automatically, cycling between approximately 94° C. (for denaturation), 55° C. (for oligonucleotide annealing), and 72° C. (for synthesis). Other reagents required for amplification of a PCR product include a DNA polymerase (such as Taq or Pfu polymerase), each of the four nucleotide dNTP building blocks of DNA in equimolar amounts (50-200 μM) and a buffer appropriate for the enzyme optimised for Mg2+ concentration (0.5-5 mM).

The amplification product is cloned into pCR 4 using either, TOPO TA cloning for Taq PCR products or Zero Blunt TOPO cloning for Pfu PCR products (both kits commercially available from Invitrogen). The resultant clone is checked by sequencing. Any additional restriction sequences which are not compatible with the cloning system are then removed using site directed mutagenesis (for example using Quickchange (Stratagene Inc.)).

### Preparation of H_{N}/C Insert

The H_{N} is created by one of two ways:

The DNA sequence is designed by back translation of the H_{N}/C amino acid sequence (obtained from freely available database sources such as GenBank (accession number P18640) or Swissprot (accession locus BXC1_CLOBO)) using one of a variety of reverse translation software tools (for example EditSeq best E. coli reverse translation (DNASTAR Inc.), or Back translation tool v2.0 (Entelechon)). A PstI restriction sequence added to the N-terminus and XbaI-stop codon-HindIII to the C-terminus ensuring the correct reading frame in maintained. The DNA sequence is screened (using software such as MapDraw, DNASTAR Inc.) for restriction enzyme cleavage sequences incorporated during the back translation. Any sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector.

The alternative method is to use PCR amplification from an existing DNA sequence with PstI and XbaI-stop codon-HindIII restriction enzyme sequences incorporated into the 5′ and 3′ PCR primers respectively. The PCR amplification is performed as described above. The PCR product is inserted into pCR 4 vector and checked by sequencing. Any additional restriction sequences which are not compatible with the cloning system are then removed using site directed mutagenesis (for example using Quickchange (Stratagene Inc.)).

### Preparation of the Spacer (LC-H_{N} Linker)

The LC-H_{N} linker can be designed from first principle, using the existing sequence information for the linker as the template. For example, the serotype C linker (in this case defined as the inter-domain polypeptide region that exists between the cysteines of the disulphide bridge between LC and H_{N}) has the sequence HKAIDGRSLYNKTLD. This sequence information is freely available from available database sources such as GenBank (accession number P18640) or Swissprot (accession locus BXC1_CLOBO). For generation of a specific protease cleavage site, the recognition sequence for enterokinase is inserted into the activation loop to generate the sequence VDGIITSKTKSDDDDKNKALNLQ. Using one of a variety of reverse translation software tools (for example EditSeq best E. coli reverse translation (DNASTAR Inc.), or Backtranslation tool v2.0 (Entelechon)), the DNA sequence encoding the linker region is determined. BamHI/SalI and PstI/XbaI/stop codon/HindIII restriction enzyme sequences are incorporated at either end, in the correct reading frames. The DNA sequence is screened (using software such as MapDraw, DNASTAR Inc.) for restriction enzyme cleavage sequences incorporated during the back translation. Any sequences that are found to be common to those required by the cloning system are removed manually from the proposed coding sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector. If it is desired to clone the linker out of pCR 4 vector, the vector (encoding the linker) is cleaved with either BamHI+SalI or PstI+XbaI combination restriction enzymes. This cleaved vector then serves as the recipient vector for insertion and ligation of either the LC DNA (cleaved with BamHI/SalI) or H_{N} DNA (cleaved with PstI/XbaI). Once the LC or the H_{N} encoding DNA is inserted upstream or downstream of the linker DNA, the entire LC-linker or linker-H_{N} DNA fragment can the be isolated and transferred to the backbone clone.

As an alternative to independent gene synthesis of the linker, the linker-encoding DNA can be included during the synthesis or PCR amplification of either the LC or H_{N}.

### Assembly and Confirmation of the Backbone Clone

The LC or the LC-linker is cut out from the pCR 4 cloning vector using BamHI/SalI or BamHI/PstI restriction enzymes digests. The pMAL expression vector is digested with the same enzymes but is also treated with calf intestinal protease (CIP) as an extra precaution to prevent re-circularisation. Both the LC or LC-linker region and the pMAL vector backbone are gel purified. The purified insert and vector backbone are ligated together using T4 DNA ligase and the product is transformed with TOP10 cells which are then screened for LC insertion using BamHI/SalI or BamHI/PstI restriction digestion. The process is then repeated for the H_{N} or linker-H_{N} insertion into the PstI/HindIII or SalI/HindIII sequences of the pMAL-LC construct.

Screening with restriction enzymes is sufficient to ensure the final backbone is correct as all components are already sequenced confirmed, either during synthesis or following PCR amplification. However, during the sub-cloning of some components into the backbone, where similar size fragments are being removed and inserted, sequencing of a small region to confirm correct insertion is required.

### Example 9

### Construction, Expression, and Purification of a LH_{N}/C-EGF Fusion Protein

### Preparation of Spacer-EGF Insert

For presentation of an EGF sequence at the C-terminus of the H_{N} domain, a DNA sequence is designed to flank the spacer and targeting moiety (TM) regions allowing incorporation into the backbone clone (SEQ ID2). The DNA sequence can be arranged as BamHI-SalI-PstI-XbaI-spacer-EGF-stop codon-HindIII (SEQ ID3). The DNA sequence can be designed using one of a variety of reverse translation software tools (for example EditSeq best E. coli reverse translation (DNASTAR Inc.), or Backtranslation tool v2.0 (Entelechon)). Once the TM DNA is designed, the additional DNA required to encode the preferred spacer is created in silico. It is important to ensure the correct reading frame is maintained for the spacer, EGF and restriction sequences and that the XbaI sequence is not preceded by the bases, TC which would result on DAM methylation. The DNA sequence is screened for restriction sequence incorporated and any additional sequences are removed manually from the remaining sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector.

Insertion of Spacer-EGF into Backbone

In order to create a LC-linker-H_{N}-spacer-EGF construct (SEQ ID4) using the backbone construct (SEQ ID2) and the newly synthesised pCR 4-spacer-TM vector encoding the EGF TM (SEQ ID3), the following two-step method is employed. Firstly, the H_{N} domain is excised from the backbone clone using restriction enzymes PstI and XbaI and ligated into similarly digested pCR 4-spacer-EGF vector. This creates an H_{N}-spacer-EGF ORF in pCR 4 that can be excised from the vector using restriction enzymes PstI and HindIII for subsequent ligation into similarly cleaved backbone or expression construct. The final construct contains the LC-linker-H_{N}-spacer-EGF ORF (SEQ ID4) for transfer into expression vectors for expression to result in a fusion protein of the sequence illustrated in SEQ ID5.

Screening with restriction enzymes is sufficient to ensure the final backbone is correct as all components are already sequenced confirmed, either during synthesis or following PCR amplification. However, during the sub-cloning of some components into the backbone, where similar size fragments are being removed and inserted, sequencing of a small region to confirm correct insertion is required.

### Alternative Construction Approach

As an alternative to the methodologies described above for the construction of LH_{N}/C-EGF, complete gene synthesis has been used to create a single DNA insert that encodes the LC, the H_{N}, linkers, spacers and a protease activation site. The synthetic DNA is designed to have a NdeI restriction site at the 5′ end and a HindIII restriction site at the 3′ end to facilitate direct cloning into expression vectors. The sequence of the engineered coding region is subject to the same codon utilisation analysis as described above. The sequence of the synthetic DNA is illustrated in SEQ ID19, and the protein that it encodes is illustrated in SEQ ID20.

### Expression of LH_{N}/C-EGF Fusion Protein

Expression of the LH_{N}/C-EGF fusion protein is achieved using the following protocol. Inoculate 100 ml of modified TB containing 0.2% glucose and 100 μg/ml ampicillin in a 250 ml flask with a single colony from the LH_{N}/C-EGF expression strain. Grow the culture at 37° C., 225 rpm for 16 hours. Inoculate 1 L of modified TB containing 0.2% glucose and 100 μg/ml ampicillin in a 2 L flask with 10 ml of overnight culture. Grow cultures at 37° C. until an approximate OD600 nm of 0.5 is reached at which point reduce the temperature to 16° C. After 1 hour induce the cultures with 1 mM IPTG and grow at 16° C. for a further 16 hours.

### Purification of LH_{N}/C-EGF Fusion Protein

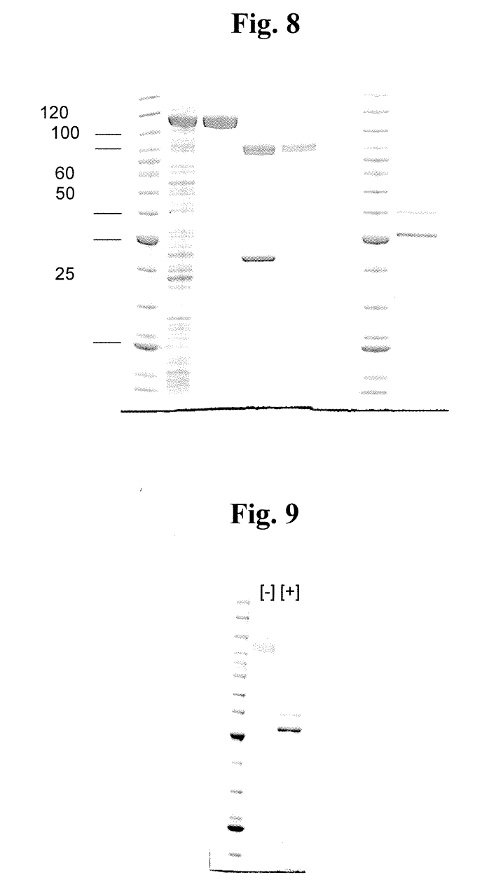

Defrost falcon tube containing 25 ml 50 mM HEPES pH 7.2 200 mM NaCl and approximately 10 g of E. coli BL21 cell paste. Sonicate the cell paste on ice 30 seconds on, 30 seconds off for 10 cycles at a power of 22 microns ensuring the sample remains cool. Spin the lysed cells at 18 000 rpm, 4° C. for 30 minutes. Load the supernatant onto a 0.1 M NiSO₄ charged Chelating column (20-30 ml column is sufficient) equilibrated with 50 mM HEPES pH 7.2 200 mM NaCl. Using a step gradient of 10 and 40 mM imidazole, wash away the non-specific bound protein and elute the fusion protein with 100 mM imidazole. Dialyse the eluted fusion protein against 5 L of 50 mM HEPES pH 7.2 200 mM NaCl at 4° C. overnight and measure the OD of the dialysed fusion protein. Add 1 unit of factor Xa per 100 μg fusion protein and incubate at 25° C. static overnight. Load onto a 0.1 M NiSO₄ charged Chelating column (20-30 ml column is sufficient) equilibrated with 50 mM HEPES pH 7.2 200 mM NaCl. Wash column to baseline with 50 mM HEPES pH 7.2 200 mM NaCl. Using a step gradient of 10 and 40 mM imidazole, wash away the non-specific bound protein and elute the fusion protein with 100 mM imidazole. Dialyse the eluted fusion protein against 5 L of 50 mM HEPES pH 7.2 200 mM NaCl at 4° C. overnight and concentrate the fusion to about 2 mg/ml, aliquot sample and freeze at −20° C. Test purified protein using OD, BCA and purity analysis. FIG. 8 demonstrates the purified protein as analysed be SDS-PAGE.

### Example 10

### Construction, Expression and Purification of a LH_{N}/B-EGF Fusion Protein

The LC-H_{N} linker is designed using the methods described in example 9 using the B serotype linker arranged as BamHI-SalI-PstI-XbaI-spacer-EGF-stop codon-HindIII (SEQ ID3). The LH_{N}/B-EGF fusion is then assembled using the LH_{N}/B backbone clone (SEQ ID1) made using the methods described in example 7 and constructed using methods described in example 9. The final construct contains the LC-linker-H_{N}-spacer-EGF ORF (SEQ ID6) for transfer into expression vectors for expression to result in a fusion protein of the sequence illustrated in SEQ ID7. The resultant expression plasmid, pMAL LH_{N}/B-EGF is transformed into E. coli BL21 for recombinant protein expression. Expression and purification of the fusion protein was carried out as described previously except that enterokinase replaced factor Xa in the activation of the fusion protein. FIG. 9 demonstrates the purified protein as analysed by SDS-PAGE.

### Example 11

### Preparation and Purification of a LH_{N}/C-RGD Fusion Protein

### Preparation of Spacer-RGD Insert

For presentation of an RGD sequence at the C-terminus of the H_{N} domain, a DNA sequence is designed to flank the spacer and TM regions allowing incorporation into the backbone clone (SEQ ID2). The DNA sequence can be arranged as BamHI-SalI-PstI-XbaI-spacer-SpeI-RGD-stop codon-HindIII (SEQ ID8). The DNA sequence can be designed using one of a variety of reverse translation software tools (for example EditSeq best E. coli reverse translation (DNASTAR Inc.), or Backtranslation tool v2.0 (Entelechon)). Once the TM DNA is designed, the additional DNA required to encode the preferred spacer is created in silico. It is important to ensure the correct reading frame is maintained for the spacer, RGD and restriction sequences and that the XbaI sequence is not preceded by the bases, TC which would result on DAM methylation. The DNA sequence is screened for restriction sequence incorporated and any additional sequences are removed manually from the remaining sequence ensuring common E. coli codon usage is maintained. E. coli codon usage is assessed by reference to software programs such as Graphical Codon Usage Analyser (Geneart), and the % GC content and codon usage ratio assessed by reference to published codon usage tables (for example GenBank Release 143, Sep. 13, 2004). This optimised DNA sequence is then commercially synthesized (for example by Entelechon, Geneart or Sigma-Genosys) and is provided in the pCR 4 vector.

Insertion of Spacer-RGD into Backbone

In order to create a LC-linker-H_{N}-spacer-RGD construct (SEQ ID9) using the backbone construct (SEQ ID2) and the newly synthesised pCR 4-spacer-TM vector encoding the RGD TM (SEQ ID8), the following two-step method is employed. Firstly, the H_{N} domain is excised from the backbone clone using restriction enzymes PstI and XbaI and ligated into similarly digested pCR 4-spacer-RGD vector. This creates an H_{N}-spacer-RGD ORF in pCR 4 that can be excised from the vector using restriction enzymes PstI and HindIII for subsequent ligation into similarly cleaved backbone or expression construct. The final construct contains the LC-linker-H_{N}-spacer-RGD ORF (SEQ ID9) for transfer into expression vectors for expression to result in a fusion protein of the sequence illustrated in SEQ ID10.

Screening with restriction enzymes is sufficient to ensure the final backbone is correct as all components are already sequenced confirmed, either during synthesis or following PCR amplification. However, during the sub-cloning of some components into the backbone, where similar size fragments are being removed and inserted, sequencing of a small region to confirm correct insertion is required.

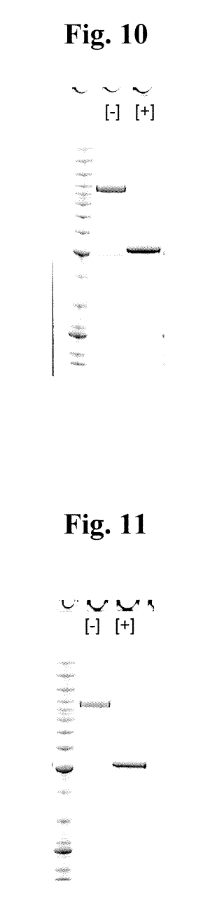

Expression and purification of the fusion protein was carried out as described in example 9. FIG. 10 demonstrates the purified protein as analysed by SDS-PAGE.

### Example 12

### Preparation and Purification of a LH_{N}/C-Cyclic RGD Fusion Protein

The LC-H_{N} linker can be designed using the methods described in example 11 using the C serotype linker arranged as BamHI-SalI-PstI-XbaI-spacer-SpeI-cyclic RGD-stop codon-HindIII (SEQ ID11). The LH_{N}/C-cyclic RGD fusion is then assembled using the LH_{N}/C backbone clone (SEQ ID2) made using the methods described in example 8 and constructed using methods described in example 11. The final construct contains the LC-linker-H_{N}-spacer-cyclic RGD ORF (SEQ ID11) for transfer into expression vectors for expression to result in a fusion protein of the sequence illustrated in SEQ ID13. The resultant expression plasmid, pMAL LH_{N}/C-cyclic RGD was transformed into E. coli BL21 for recombinant protein expression. Expression and purification of the fusion protein was carried out as described in example 9. FIG. 11 demonstrates the purified protein as analysed by SDS-PAGE.

### Example 13

### Preparation and Purification of a LC/C-RGD-H_{N}/C Fusion Protein

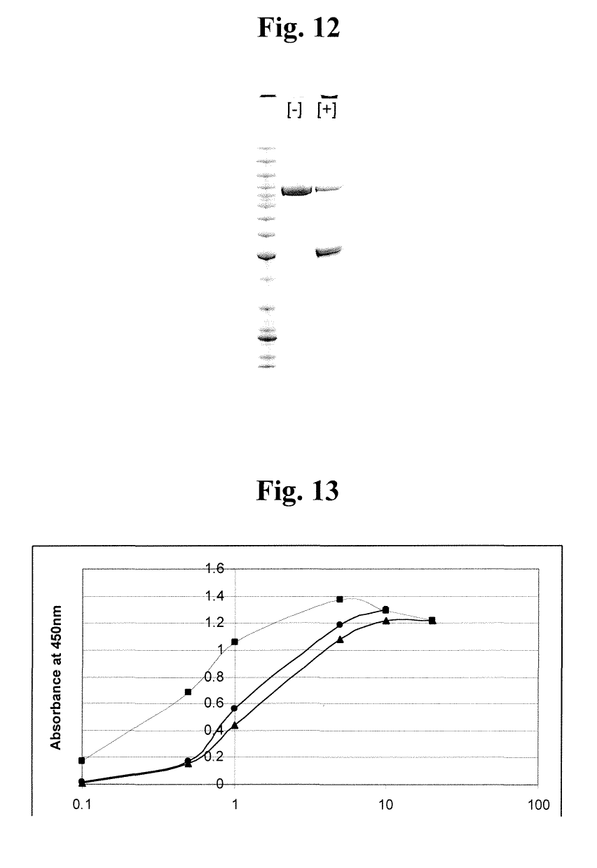

In order to create the LC-linker-RGD-spacer-H_{N} construct (SEQ ID15), the pCR 4 vector encoding the linker (SEQ ID14) is cleaved with BamHI+SalI restriction enzymes. This cleaved vector then serves as the recipient vector for insertion and ligation of the LC/C DNA (SEQ ID2) cleaved with BamHI+SalI. The resulting plasmid DNA is then cleaved with PstI+XbaI restriction enzymes and serves as the recipient vector for the insertion and ligation of the H_{N}/C DNA (SEQ ID2) cleaved with PstI+XbaI. The final construct contains the LC-linker-RGD-spacer-H_{N} ORF (SEQ ID15) for transfer into expression vectors for expression to result in a fusion protein of the sequence illustrated in SEQ ID16. The resultant expression plasmid, pMAL LC/C-RGD-H_{N}/C was transformed into E. coli BL21 for recombinant protein expression. Expression and purification of the fusion protein was carried out as described in example 9. FIG. 12 demonstrates the purified protein as analysed by SDS-PAGE.

### Alternative Construction Approach

As an alternative to the methodologies described above for the construction of LC-linker-RGD-spacer-H_{N}, complete gene synthesis has been used to create a single DNA insert that encodes the LC, the H_{N}, linkers, spacers and a protease activation site. The synthetic DNA is designed to have a NdeI restriction site at the 5′ end and a HindIII restriction site at the 3′ end to facilitate direct cloning into expression vectors. The sequence of the engineered coding region is subject to the same codon utilisation analysis as described above. The sequence of the synthetic DNA is illustrated in SEQ ID17, and the protein that it encodes is illustrated in SEQ ID18.

### Example 14

### VAMP Cleavage Activity Assay

A range of concentrations of LH_{N}/B-EGF in cleavage buffer (50 mM HEPES pH7.4, 10 mM DTT, 20 μM ZnCl₂, 1% FBS) are incubated with biotinylated VAMP substrate (1 mg/ml) for two hours at 37° C. in a shaking incubator. The cleavage reaction is transferred to a washed 96-well streptavidin coated plate and incubated at 37° C. in a shaking incubator for 5 minutes. The plate is washed three times with PBS-0.1% tween-20 (PBS-T). The wells are blocked with blocking buffer (5% FCS in PBS-T) for 1 hour at 37° C. The primary antibody (anti-FESS) is added at a dilution of 1 in 500 in blocking buffer and the plate is incubated at 37° C. for 1 hour. The plate is washed three times with PBS-T and the secondary antibody (anti guinea pig HRP conjugate) diluted 1 in 1000 in blocking buffer is applied. Following 1 hour incubation at 37° C. the plate is developed with bioFX TMB substrate. Colour development is allowed to proceed for 1-5 minutes and then stopped with stop solution. The absorbance is measured at 450 nm. FIG. 13 shows the VAMP cleavage activity of LH_{N}/B-EGF fusion protein.

### Example 15

### Effect of EGF-LH_{N}/C on Secreting Cell Lines

### Cell Cultivation

NCI-H292 (human mucoepimodial) cells were seeded into six well plates (Costar) four days prior to use from stocks grown in large 75 cm² flasks (Nunc). The cells were cultured in RPMI+10% foetal bovine serum (FBS)+2 mM L-glutamine glutamine in a humidified 5% CO₂ incubator at 37° C. A549 (human lung adencocarcinoma) cells were seeded into six well plates (Costar) four days prior to use from stocks grown in large 75 cm² flasks (Nunc). The cells were cultured in DMEM+10% FBS+2 mM L-glutamine in a humidified 5% CO₂ incubator at 37° C. In both cases the cells were used at 80% confluence in the wells. Prior to all treatments, the cells were serum-starved for 24 hours.

### Inhibition of Mucin Release Following Application of EGF-LH_{N}/C

Both NCI-H292 and A549 cells were treated with various concentrations of fusion molecule for 24 hours in serum-free medium for the respective cell line in a humidified 5% CO₂ incubator at 37° C. The media was removed and fresh serum-free medium containing the fusion molecule was added and the cells incubated plus the stimulant EGF/TNF alpha (20 ng/ml and 25 ng/ml respectively) for a further 48 hours and the cells incubated in a humidified 5% CO₂ incubator at 37° C. Following stimulation, the media were collected, stored at −20° C. and analysed for mucin content using an enzyme-linked lectin assay (ELLA).

### Detection of Released Mucus in Cell Culture Medium

Lectin derived from Helix pomatia (edible snail), which binds to specific terminal sugars in mucin glycoproteins, was adhered to a ninety-six well Maxisorp™ plate in phosphate buffered saline (PBS) pH 6.8 for one hour at 37° C. Excess PBS plus lectin were removed from the plate by washing with 0.5 M NaCl, PBS, 0.1% Tween 20, pH 7.4. Cell culture medium collected from treated cells was added and the plate incubated for one hour at 37° C. The plates were then washed in 0.5 M NaCl, PBS, 0.1% Tween 20, pH 7.4 before adding Horseradish Peroxidase conjugated Helix pomatia lectin in PBS pH 7.4. Following a further wash step, the assay was developed using OPD (o-phenylenediamine dihydrochloride) peroxidase substrate for ten minutes at 37° C. The reaction was terminated with 2% H₂SO₄ (final concentration) and the absorbance at 492 nm read using a plate reader. Standard curves using human mucin prepared from sputum samples according to literature precedence and medium only controls were included on each Maxisorp™ plate.

### Detection of Mucin Release and Cell Mucin Content Using Western Blotting Technique

Cells treated with EGF-LH_{N}/C and stimulated were lysed in buffer and run on an agarose gel, transferred to nitrocellulose membranes and probed with Muc5AC monoclonal antibodies (Kirkham, et al 2002 Biochem. J. 361: pp 537). The bands were detected using secondary antibodies conjugated to horseradish peroxidase and the resultant band densities were scanned. Media from treated cells was also analysed using this method.

### Cell Viability Assay

Cells were cultured in 6 well plates, the supernatant was removed and MTT (3-[4,5-dimethylthiazol-2-yl]-2,5-diphenyltetrazolium bromide) was added per well and incubated until a purple tinge was visible. The MTT was removed, and DMSO was added to solubilize the cells and the absorbance read at 550 nm. Results are expressed as percentage baseline control viability with control set at 100% (Mosmann 1983).

### Inhibition of Mucus Release Following Application of EGF-LH_{N}/C

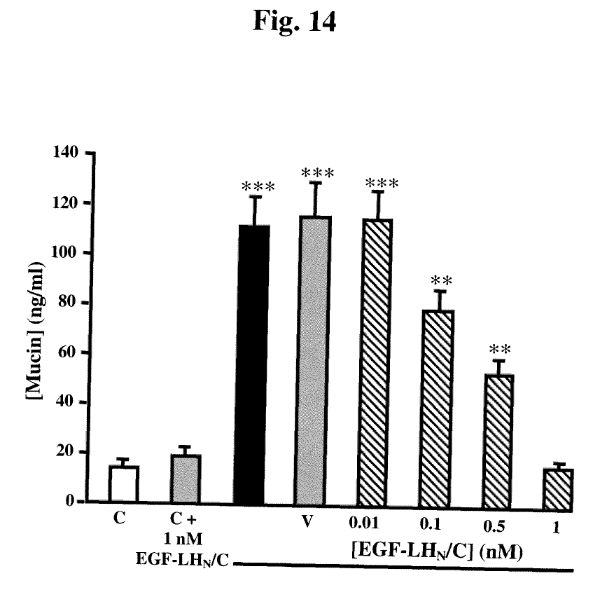

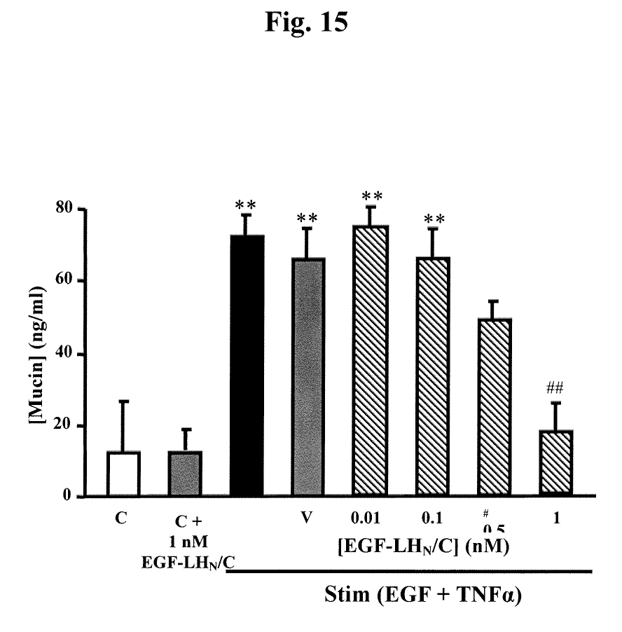

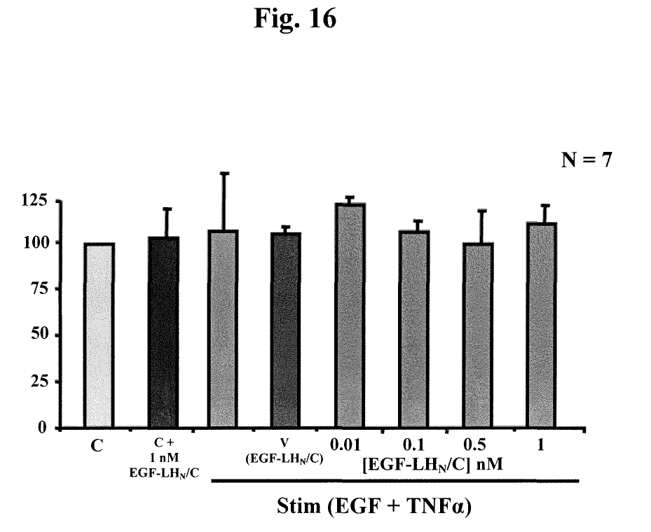

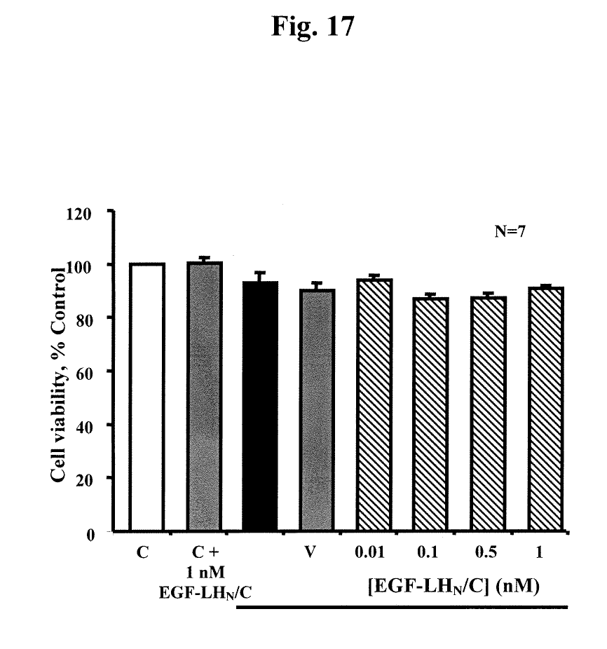

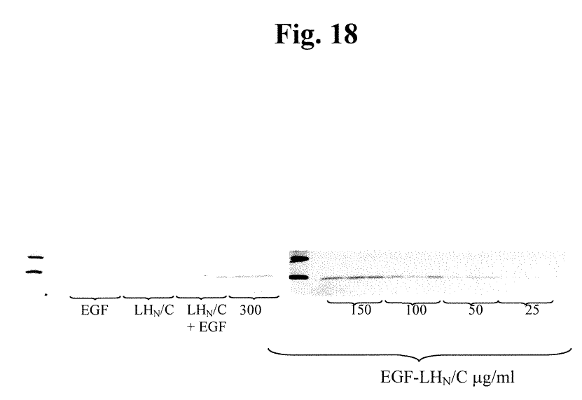

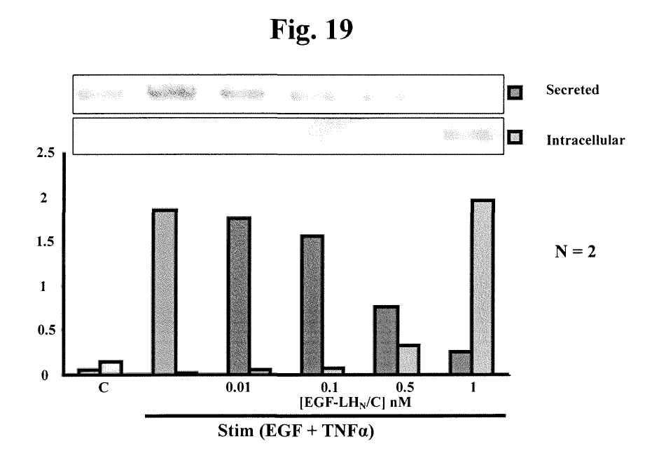

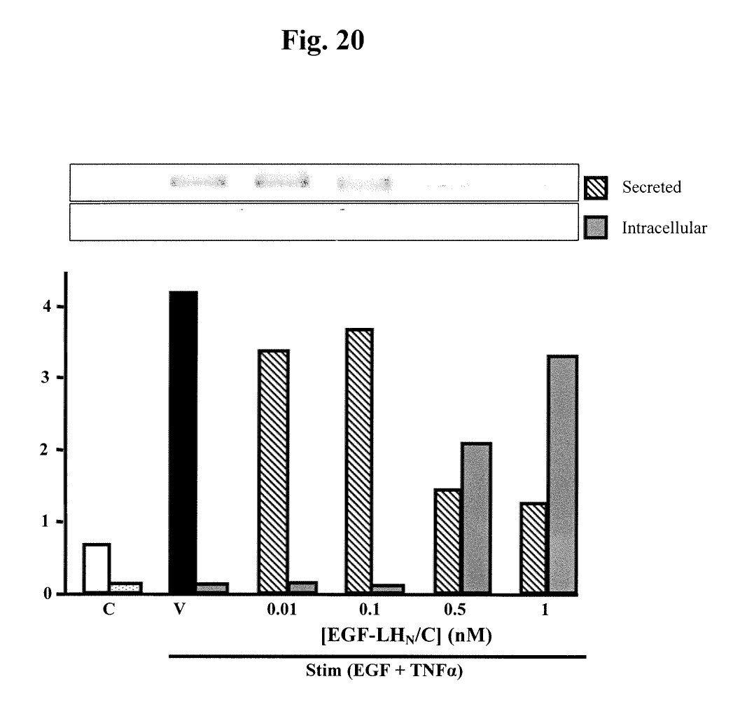

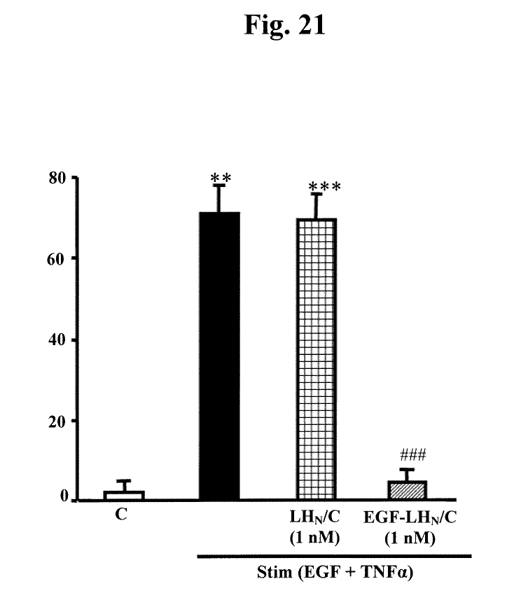

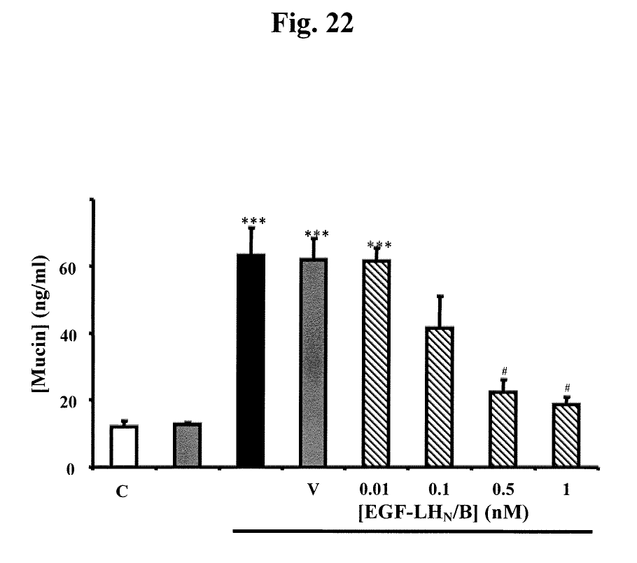

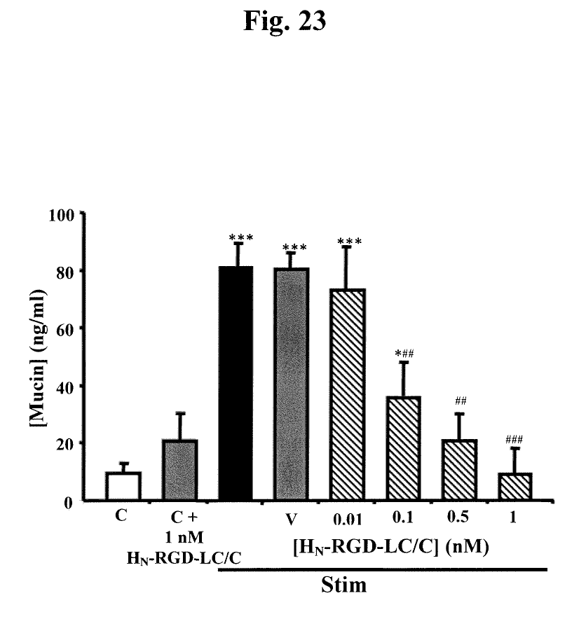

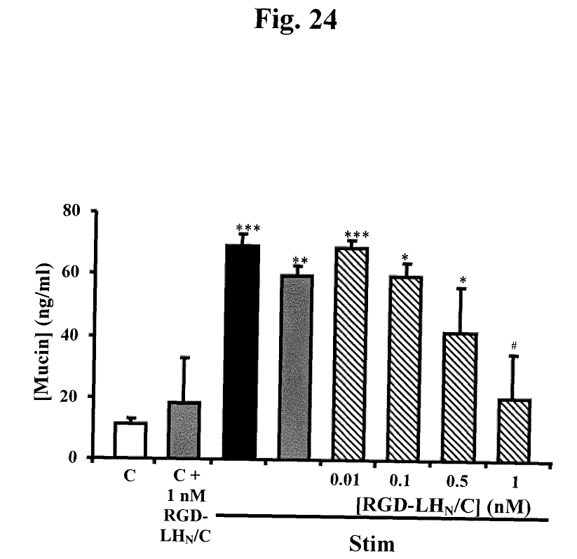

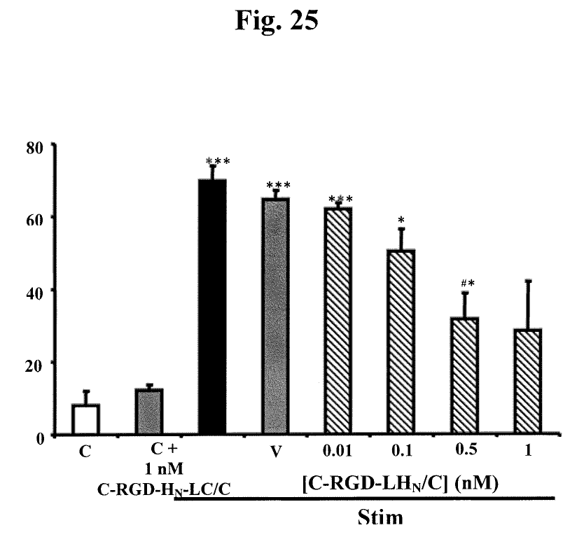

FIG. 14 shows a dose-response curve of mucus release from A549 cells. The figure shows the clear inhibition of mucus release from these cells following treatment with an IC50 of 0.4 nM. Data shown is from seven independent experiments. FIG. 15 shows that the fusion EGF-LH_{N}/C inhibited mucin release in a dose-dependent manner from NCI-H292 cells. Data from two different cell lines indicates that the fusion functions as proposed and blocks secretion of mucin from the cells.

EGF-LH_{N}/C does not Affect Viability of A549 or NCI-H292 Cells

FIGS. 16 and 17 show that cell viability as measured by MTT assay is not affected by EGF-LH_{N}/C, indicating that reduction of mucin secretion is not due to cell death.

### Effect of EGF-LH_{N}/C on Mucin Content in Treated Cells

EGF-LH_{N}/C blocks release of mucin from stimulated cells (FIGS. 14 & 15) through cleavage of the SNARE protein syntaxin (FIG. 18). Concomitant with a decrease in mucin release as the EGF-LH_{N}/C concentration increases is an increase in the amount of mucin detected in the treated cells. FIG. 19 shows from two independent experiments that as level of MUC5AC remaining in a treated cell (intracellular mucin) increases as the EGF-LH_{N}/C concentration increases. FIG. 20 shows a similar result but with MUC5B.

### Example 13

### In Vitro Cleavage of a SNARE Protein by EGF-LH_{N}/C

Cleavage of the SNARE Mediated Protein in Cells Following Treatment with EGF-LH_{N}/C

The fusion EGF-LH_{N}/C can obtain entry to cells and traffic to the SNARE proteins located near the plasma membrane. Cleavage of the SNARE protein Syntaxin by the EGF-LH_{N}/C fusion inhibits release of mucin from the cell. FIG. 18 shows syntaxin was cleaved by EGF-LH_{N}/C in a concentration-dependent manner in NCI-H292 cells. Cells were treated with 300 μg/ml EGF and/or 300 μg/ml LH_{N}/C or various concentrations of EGF-LH_{N}/C in serum-free medium for 3 days. Membrane proteins were extracted using chloroform/methanol and analysed by western blotting using a rabbit polyclonal antibody raised against the cleavage site peptide sequence of the smaller C-terminal syntaxin cleavage product, AVKY, as described previously (Sutton et al., 2005 Prot. Purif. Exp. 31:pp 41). Neither EGF alone, nor LH_{N}/C alone nor EGF and LH_{N}/C in combination were able to cleave syntaxin in the intact cells. These results confirm that the recombinant EGF-LH_{N}/C possesses a functional endopeptidase activity and is able to gain access to the interior of intact NCI-H292 cells.

### Example 14

### Effect of LH_{N}/C on Stimulated Mucin Secretion

Effect of LH_{N}/C on Mucin Release from A549 Cells

FIG. 21 shows that treatment of A549 cells with LH_{N}/C had no effect on the release of detectable mucin indicating the requirement for the ligand, in this case EGF, to allow the fusion to gain access to the cell and the SNARE mediated secretion apparatus.

Cells treated for 24 hr with 1 nM LH_{N}/C then stimulated for 48 hr with EGF/TNF alpha in the continued presence of LH_{N}/C showed no inhibition in mucin secretion as measured using the ELLA. Cells treated 24 hr with EGF-LH_{N}/C then stimulated for 48 hr with EGF/TNF alpha in the presence of EGF-LH_{N}/C showed inhibition of mucin secretion in the same experiment.

### Example 15

### Effect of EGF-LH_{N}/C on Baseline Secretion

FIGS. 14 and 15 show that treatment of cells for 72 hours with 1 nm EGF-does not affect baseline secretion in either A549 cells or NCI-H292 cells.

### Example 16

### Effect of EGF-LH_{N}/B on Secreting Cells

See example 15 for cell cultivation and released mucin detection.

Inhibition of Mucin Release from A549 Cells Following Treatment with EGF-LH_{N}/B

FIG. 22 shows a dose-response curve for EGF-LH_{N}/B in A549 cells. There is an inhibition of mucin release from these cells following treatment. The cells were treated with the fusion or vehicle or medium alone for 48 hour followed by a 24 hour stimulation of the cells with EGF/TNF alpha (Stimulus) in the presence of the fusion. Vehicle treated cells received fresh vehicle plus medium only.

### Example 17

### Effect of LC/C-RGD-LH_{N}/C on Secreting Cells

See example 15 for cell cultivation and mucin detection

Inhibition of Mucin Release from A549 Cells Following Treatment with LC/C-RGD-LH_{N}/C

A549 cells were treated for 48 hours with various concentrations of LC/C-RGD-LH_{N}/C followed by stimulation of cells using EGF and TNF alpha (20 ng/ml and 25 ng/ml respectively) for 24 hours in the presence of LC/C-RGD-LH_{N}/C. FIG. 23 shows a dose-dependent decrease in mucin release. The Integrin-binding ligand sequence enables the LH_{N}/C to enter the cells thereby inhibiting mucin release.

### Example 18

### Effect of RGD-LH_{N}/C on Secreting Cells

Inhibition of Mucin Release from A549 Cells Following Treatment with RGD-LH_{N}/C

A549 cells were treated for 48 hours with various concentrations of RGD-LH_{N}/C followed by stimulation of cells using EGF and TNF alpha (20 ng/ml and 25 ng/ml respectively) for 24 hours in the presence of RGD-LH_{N}/C. FIG. 24 shows a dose-dependent decrease in mucin release. The Integrin-binding ligand sequence enables the LH_{N}/C to enter the cells thereby inhibiting mucin release.

### Example 19

### Effect of LH_{N}/C-Cyclic-RGD on Secreting Cells

Inhibition of Mucin Release from A549 Cells Following Treatment with LH_{N}/C-Cyclic-RGD

A549 cells were treated for 48 hours with various concentrations of LH_{N}/C-cyclic-RGD followed by stimulation of cells using EGF and TNF alpha (20 ng/ml and 25 ng/ml respectively) for 24 hours in the presence of LH_{N}/C-cyclic-RGD. FIG. 25 shows a dose-dependent decrease in mucin release. The Integrin-binding ligand sequence enables the LH_{N}/C to enter the cells thereby inhibiting mucin release.

### Example 20

### Other Growth Factors as Ligands for Fusion Molecules that Inhibit Secretion from Mucous-Secreting Cells

Vascular Endothelial Growth Factor

Platelet-Derived Growth Factor

Keratinocyte Growth Factor

Hepatocyte Growth Factor

Transforming Growth Factor—alpha

Transforming Growth Factor—beta

### Example 21

### Other Peptides as Ligands for Fusion Molecules that Inhibit Secretion from Mucous-Secreting Cells

Atrial Natriuretic Peptide as a ligand to create ANP-LH_{N}/C: Ligand receptor is found in lung. Ligand-receptor become internalised. High receptor affinity.

Vasoactive Intestinal Polypeptide as a ligand to create VIP-LH_{N}/C: Good receptor affinity. Receptor expressed in tracheal and bronchial epithelial cells and mucous cells of submucosal gland.

Phage display peptide THALW(H)T as a ligand to create THALW(H)T-LH_{N}/C: THAL(H)WT binds to apical surface of human bronchial and tracheal epithelial cells.

### Example 22

### Other Integrin-Binding Ligands as Ligands for Clostridial Endopeptidase-Based Fusion Molecules that Inhibit Secretion from Mucous-Secreting Cells

Multivalent RGD motif as a ligand to create mvRGD-LH_{N}/C: High ligand receptor affinity. Integrin targeted is highly lung-specific and expressed on human bronchial epithelial cells.

GRGDSP

GRGESP

Vitronectin

Fibronectin

Lamimin

Fibrinogen

Heparin

Phytohaemagglutinin

### Example 23

### Clinical Example

A 56 year old male suffering from chronic bronchitis (FEV1 reduced to 80% of normal predicted value; daily sputum volume of 30 ml) presents at his GP. Despite treatment with inhaled steroids, the patient presents with difficulty in performing everyday tasks due continued shortness of breath. The GP prescribes a 6-month course of EGF-LH_{N}/C (as prepared in Example 11) in nebuliser form, 80 μg to be taken monthly. Following discussion with the physician, the patient selects the most appropriate nebuliser for their personal situation from a range of suitable devices. After a single dose of EGF-LH_{N}/C the patient experiences reduced sputum volume (to 15 ml) and an improvement in FEV1 (to 90%). Further treatment enhances these parameters further and improves quality of life.

### Example 24

### Clinical Example

A 14 year old female suffering from cystic fibrosis presents at her GP. Despite treatment with pulmozyme and extensive physiotherapy, the patient's respiratory performance (as measured by FEV1) continues to decline. The GP prescribes a 6-month course of RGD peptide-LH_{N}/C (as prepared in Example 13) in nebuliser form, 80 μg to be taken monthly immediately after physiotherapy. Following discussion with the physician, the patient selects the most appropriate nebuliser for their personal situation from a range of suitable devices. After a single dose of RGD peptide-LH_{N}/C the patient experiences an improvement in FEV1. Further treatment enhances respiratory performance further and improves quality of life.

### Example 25

An agent of the present invention can be prepared by conventional chemical conjugation techniques, or by recombinant technology, thereby providing an agent comprising a cytotoxin selected from the list below (or a fragment or variant thereof that substantially retains the cytotoxic activity thereof); a translocation domain of a clostridial neurotoxin or an alternative translocation domain (eg. as listed below); and a suitable TM.

### Cytotoxins:

VacA cytotoxin

Exotoxin A (ETA)

Pertussis toxin (PT)

Adenylate cyclase toxin (CYA)

Heat labile enterotoxin (LT)

Cholera toxin

EDIN

Exoenzyme C3

Diphtheria toxin

Shiga-toxin A chain

Shiga-like toxin A chain

α-sarcin

Cytotoxins C1 and C2

Cytotoxic phospholipase A2

IRIP type I ribosome inactivating protein

Cinnamomin type II ribosome inactivating protein

Iris agglutinins b and r

Ricin A-chain

Abrin A-chain

Pokeweed antiviral protein

Mistletoe lectin A subunit

Saporin 6

Wheatgerm agglutinin A chain

Dianthin 30

Gelonin

Trichosanthin

### Translocation Domains:

Diphtheria toxin translocation domain

Domain II of Pseudomonas exotoxin

Influenza virus haemagglutinin

Semliki Forest Virus fusogenic protein translocation domain

Vesicular Stomatitis virus glycoprotein G

SER virus F protein translocation domain

Foamy virus envelope glycoprotein translocation domain

### Example 26

### Preparation of VacA Cytotoxin-Epidermal Growth Factor with a Translocation Domain from Diphtheria Toxin by Recombinant Expression

Using standard DNA manipulation procedures, the DNA encoding VacA cytotoxin, diphtheria toxin amino acids 194-380, and epidermal growth factor are assembled in frame and inserted into an appropriate expression vector. Inserted between the VacA cytotoxin DNA and the diphtheria toxin translocation domain is DNA encoding a short spacer sequence with a specific cleavable peptide bond (↓), bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form VacA cytotoxin-DT.sub.194-380-EGF is achieved in E. coli using standard techniques. The expressed fusion protein is isolated from E. coli by standard purification techniques and cleaved by the specific activation enzyme prior to assessment in an in vitro cell model.

### Example 27

### Preparation of Exotoxin A-Epidermal Growth Factor with a Translocation Domain from Pseudomonas Exotoxin by Recombinant Expression

Using standard DNA manipulation procedures, the DNA encoding Exotoxin A, pseudomonas exotoxin amino acids 405-613, and epidermal growth factor are assembled in frame and inserted into an appropriate expression vector. Inserted between the Exotoxin A DNA and the pseudomonas exotoxin translocation domain is DNA encoding a short spacer sequence with a specific cleavable peptide bond, bounded by a pair of cysteine amino acids. Examples of specific enzymes that may be used to activate the fusion protein include factor Xa (IEGR↓), (SEQ ID NO: 24), enterokinase (DDDDK↓) (SEQ ID NO:25), TEV protease (EXXYXQS↓G) (SEQ ID NO: 26), precission (LEVLFQ↓GP) (SEQ ID NO: 27), Thrombin (LVPR↓GS) (SEQ ID NO: 28) and genenase (HY or YH). Expression of a single polypeptide of the form Exotoxin A-PE.sub.405-613-EGF is achieved in E. coli using standard techniques. The expressed fusion protein is isolated from E. coli by standard purification techniques and cleaved by the specific activation enzyme prior to assessment in an in vitro cell model.

### Example 28

### Preparation of Pertussis Toxin-Epidermal Growth Factor with a Translocation Domain from Influenza Virus Haemagglutinin by Recombinant Expression

Using standard DNA manipulation procedures, the DNA encoding Pertussis Toxin (PT), GLFGAIAGFIENGWEGMIDGWYG (SEQ ID NO:21) from influenza virus haemagglutinin (HA), and epidermal growth factor are assembled in frame and inserted into an appropriate expression vector. Inserted between the PT DNA and the haemagglutinin sequence is DNA encoding a short spacer sequence with a specific cleavable peptide bond, bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form PT-HA-EGF is achieved in E. coli using standard techniques. The expressed fusion protein is isolated from E. coli by standard purification techniques and cleaved by the specific activation enzyme prior to assessment in an in vitro cell model.

### Example 29

### Preparation of αsarcin-Epidermal Growth Factor with a Translocation Domain from Diphtheria Toxin by Recombinant Expression

Using standard DNA manipulation procedures, the DNA encoding Aspergillus giganteus αsarcin, diphtheria toxin amino acids 194-380, and epidermal growth factor are assembled in frame and inserted into an appropriate expression vector. Inserted between the αsarcin DNA and the diphtheria toxin translocation domain is DNA encoding a short spacer sequence with a specific cleavable peptide bond (↓), bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form αsarcin-DT.sub.194-380-EGF is achieved in E. coli using standard techniques. The expressed fusion protein is isolated from E. coli by standard purification techniques and cleaved by the specific activation enzyme prior to assessment in an in vitro cell model.

### Example 30

### Preparation of Ricin A-Chain-Epidermal Growth Factor with a Translocation Domain from Pseudomonas Exotoxin by Recombinant Expression

Using standard DNA manipulation procedures, the DNA encoding Ricin A-chain, pseudomonas exotoxin amino acids 405-613, and epidermal growth factor are assembled in frame and inserted into an appropriate expression vector. Inserted between the Ricin A-chain DNA and the pseudomonas exotoxin translocation domain is DNA encoding a short spacer sequence with a specific cleavable peptide bond, bounded by a pair of cysteine amino acids. Examples of specific enzymes that may be used to activate the fusion protein include factor Xa (IEGR↓), (SEQ ID NO: 24), enterokinase (DDDDK↓) (SEQ ID NO:25), TEV protease (EXXYXQS↓G) (SEQ ID NO: 26), precission (LEVLFQ↓GP) (SEQ ID NO: 27), Thrombin (LVPR↓GS) (SEQ ID NO: 28) and genenase (HY or YH). Expression of a single polypeptide of the form Ricin A-chain-PE.sub.405-613-EGF is achieved in E. coli using standard techniques. The expressed fusion protein is isolated from E. coli by standard purification techniques and cleaved by the specific activation enzyme prior to assessment in an in vitro cell model.

### Example 31

### Preparation of Saporin 6-Epidermal Growth Factor with a Translocation Domain from Influenza Virus Haemagglutinin by Recombinant Expression

Using standard DNA manipulation procedures, the DNA encoding Saporin 6, GLFGAIAGFIENGWEGMIDGWYG (SEQ ID NO:21) from influenza virus haemagglutinin (HA), and epidermal growth factor are assembled in frame and inserted into an appropriate expression vector. Inserted between the Saporin 6 DNA and the haemagglutinin sequence is DNA encoding a short spacer sequence with a specific cleavable peptide bond, bounded by a pair of cysteine amino acids. Examples of specific enzymes that may be used to activate the fusion protein include factor Xa (IEGR↓) (SEQ ID NO:24), enterokinase (DDDDK↓) (SEQ ID NO:25), TEV protease (EXXYXQS↓G) (SEQ ID NO:26), precission (LEVLFQ↓GP) (SEQ ID NO:27), Thrombin (LVPR↓GS) (SEQ ID NO:28) and genenase (HY or YH). Expression of a single polypeptide of the form Saporin 6-HA-EGF is achieved in E. coli using standard techniques. The expressed fusion protein is isolated from E. coli by standard purification techniques and cleaved by the specific activation enzyme prior to assessment in an in vitro cell model.

### Example 32

### Construction, Expression, and Purification of a Cholera Toxin-H_{N}-EGF Fusion Protein

For construction of a Cholera toxin-H_{N}-EGF fusion protein, complete gene synthesis has been used to create a single DNA insert that encodes the Cholera toxin (ChTx), the H_{N}, linkers, spacers and a protease activation site. The synthetic DNA is designed to have a NdeI restriction site at the 5′ end and a HindIII restriction site at the 3′ end to facilitate direct cloning into expression vectors.

### Expression of Cholera Toxin-H_{N}-EGF Fusion Protein

Expression of the ChTx-H_{N}-EGF fusion protein is achieved using the following protocol. Inoculate 100 ml of modified TB containing 0.2% glucose and 100 μg/ml ampicillin in a 250 ml flask with a single colony from the Cholera toxin-H_{N}-EGF expression strain. Grow the culture at 37° C., 225 rpm for 16 hours. Inoculate 1 L of modified TB containing 0.2% glucose and 100 μg/ml ampicillin in a 2 L flask with 10 ml of overnight culture. Grow cultures at 37° C. until an approximate OD600 nm of 0.5 is reached at which point reduce the temperature to 16° C. After 1 hour induce the cultures with 1 mM IPTG and grow at 16° C. for a further 16 hours.

### Purification of Cholera Toxin-H_{N}-EGF Fusion Protein

Defrost falcon tube containing 25 ml 50 mM HEPES pH 7.2 200 mM NaCl and approximately 10 g of E. coli BL21 cell paste. Sonicate the cell paste on ice 30 seconds on, 30 seconds off for 10 cycles at a power of 22 microns ensuring the sample remains cool. Spin the lysed cells at 18,000 rpm, 4° C. for 30 minutes. Load the supernatant onto a 0.1 M NiSO₄ charged Chelating column (20-30 ml column is sufficient) equilibrated with 50 mM HEPES pH 7.2 200 mM NaCl. Using a step gradient of 10 and 40 mM imidazole, wash away the non-specific bound protein and elute the fusion protein with 100 mM imidazole. Dialyse the eluted fusion protein against 5 L of 50 mM HEPES pH 7.2 200 mM NaCl at 4° C. overnight and measure the OD of the dialysed fusion protein. Add 1 unit of factor Xa per 100 μg fusion protein and incubate at 25° C. static overnight. Load onto a 0.1 M NiSO₄ charged Chelating column (20-30 ml column is sufficient) equilibrated with 50 mM HEPES pH 7.2 200 mM NaCl. Wash column to baseline with 50 mM HEPES pH 7.2 200 mM NaCl. Using a step gradient of 10 and 40 mM imidazole, wash away the non-specific bound protein and elute the fusion protein with 100 mM imidazole. Dialyse the eluted fusion protein against 5 L of 50 mM HEPES pH 7.2 200 mM NaCl at 4° C. overnight and concentrate the fusion to about 2 mg/ml, aliquot sample and freeze at −20° C. Test purified protein using OD, BCA and purity analysis.

### Example 33

A 66 year old male suffering from chronic bronchitis (FEV1 reduced to 75% of normal predicted value; daily sputum volume of 25 ml) presents at his GP. Despite treatment with inhaled steroids, the patient presents with difficulty in performing everyday tasks due continued shortness of breath. The GP prescribes a 6-month course of the fusion protein consisting of VacA cytotoxin, diphtheria toxin translocation domain and EGF TM (as prepared in Example 26) in nebuliser form, 80 μg to be taken monthly. Following discussion with the physician, the patient selects the most appropriate nebuliser for their personal situation from a range of suitable devices. After a single dose of the VacA-diphtheria toxin translocation domain-EGF fusion protein the patient experiences reduced sputum volume (to 10 ml) and an improvement in FEV1 (to 85%). Further treatment enhances these parameters further and improves quality of life.

### Example 34

A 40 year old male suffering from COPD presents at his GP. Treatment with standard bronchodilatory therapies has provided only minimal reversal of his impaired airway function. The GP prescribes a 6-month course of the fusion protein consisting of Exotoxin A DNA, pseudomonas exotoxin translocation domain and EGF TM (as prepared in Example 27) in nebuliser form, 80 μg to be taken monthly. Following discussion with the physician, the patient selects the most appropriate nebuliser for their personal situation from a range of suitable devices. After a single dose of the Exotoxin A-PE₄₀₅₋₆₁₃-EGF fusion protein the patient experiences reduced expectoration improved maximal airway flow and lung emptying. Further treatment enhances these parameters further and improves quality of life.

### Example 35

A 25 year old female suffering from asthma presents at her GP. Despite treatment with standard asthma therapies, the patient obtains only partial relief from acute asthmatic episodes. The GP prescribes a 6-month course of the fusion protein consisting of PT, influenza virus haemagglutinin domain and EGF TM (as prepared in Example 28) in nebuliser form, 80 μg to be taken monthly. Following discussion with the physician, the patient selects the most appropriate nebuliser for their personal situation from a range of suitable devices. After a single dose of the PT-influenza virus haemagglutinin domain-EGF fusion protein the patient experiences a decrease in the severity of acute asthmatic episodes. Further treatment enhances these parameters further and improves quality of life.

### Example 36

A 54 year old female suffering from chronic bronchitis (FEV1 reduced to 75% of normal predicted value; daily sputum volume of 25 ml) presents at her GP. Despite treatment with inhaled steroids, the patient presents with difficulty in performing everyday tasks due continued shortness of breath. The GP prescribes a 6-month course of a fusion protein consisting of αsarcin, diphtheria toxin amino acids 194-380, and EGF TM (as prepared in Example 29) in nebuliser form, 80 μg to be taken monthly. Following discussion with the physician, the patient selects the most appropriate nebuliser for their personal situation from a range of suitable devices. After a single dose of the αsarcin-DT₁₉₄₋₃₈₀-EGF fusion protein the patient experiences reduced sputum volume (to 10 ml) and an improvement in FEV1 (to 85%). Further treatment enhances these parameters further and improves quality of life.

### Example 37

A 69 year old female suffering from COPD presents at her GP. Treatment with bronchodilators has provided only minimal reversal of her impaired airway function. The GP prescribes a 6-month course of the fusion protein consisting of Ricin A-chain, pseudomonas exotoxin amino acids 405-613, and EGF TM (as prepared in Example 30) in nebuliser form, 80 μg to be taken monthly. Following discussion with the physician, the patient selects the most appropriate nebuliser for their personal situation from a range of suitable devices. After a single dose of the Ricin A-chain-PE₄₀₅₋₆₁₃-EGF fusion protein the patient experiences reduced expectoration improved maximal airway flow and lung emptying. Further treatment enhances these parameters further and improves quality of life.

### Example 38

A 14 year old male suffering from asthma presents at his GP. Despite treatment with standard asthma therapies, the patient obtains only partial relief from acute asthmatic episodes. The GP prescribes a 6-month course of the fusion protein consisting of Saporin 6, GLFGAIAGFIENGWEGMIDGWYG (SEQ ID NO:21) from influenza virus haemagglutinin (HA), and EGF TM (as prepared in Example 31) in nebuliser form, 80 μg to be taken monthly. Following discussion with the physician, the patient selects the most appropriate nebuliser for his personal situation from a range of suitable devices. After a single dose of the Saporin 6-HA-EGF fusion protein the patient experiences a decrease in the severity of acute asthmatic episodes. Further treatment enhances these parameters further and improves quality of life.

### Example 39

A 48 year old male suffering from chronic bronchitis (FEV1 reduced to 75% of normal predicted value; daily sputum volume of 25 ml) presents at his GP. Despite treatment with inhaled steroids, the patient presents with difficulty in performing everyday tasks due continued shortness of breath. The GP prescribes a 6-month course of a fusion protein consisting of Cholera toxin, clostridial neurotoxin H_{N} and EGF TM (as prepared in Example 32) in nebuliser form, 80 μg to be taken monthly. Following discussion with the physician, the patient selects the most appropriate nebuliser for their personal situation from a range of suitable devices. After a single dose of the Cholera toxin-H_{N}-EGF fusion protein the patient experiences reduced sputum volume (to 10 ml) and an improvement in FEV1 (to 85%). Further treatment enhances these parameters further and improves quality of life.

### Example 40

A 49 year old male suffering from COPD presents at his GP. Treatment with bronchodilators has provided only minimal reversal of his impaired airway function. The GP prescribes a 6-month course of a fusion protein consisting of Bordatella pertussis CYA, Vesicular Stomatitis virus glycoprotein G and EGF TM in nebuliser form, 80 μg to be taken monthly. Following discussion with the physician, the patient selects the most appropriate nebuliser for their personal situation from a range of suitable devices. After a single dose of the CYA-VSV glycoprotein G-EGF fusion protein the patient experiences reduced expectoration improved maximal airway flow and lung emptying. Further treatment enhances these parameters further and improves quality of life.

### Example 41

A 33 year old female suffering from asthma presents at her GP. Despite treatment with standard asthma therapies, the patient obtains only partial relief from acute asthmatic episodes. The GP prescribes a 6-month course of a fusion protein consisting of E. coli LT, Foamy virus envelope glycoprotein translocation domain and EGF TM in nebuliser form, 80 μg to be taken monthly. Following discussion with the physician, the patient selects the most appropriate nebuliser for his personal situation from a range of suitable devices. After a single dose of the LT-Foamy virus envelope glycoprotein translocation domain-EGF fusion protein the patient experiences a decrease in the severity of acute asthmatic episodes. Further treatment enhances these parameters further and improves quality of life.

### Example 42

A 51 year old female suffering from chronic bronchitis (FEV1 reduced to 75% of normal predicted value; daily sputum volume of 25 ml) presents at her GP. Despite treatment with inhaled steroids, the patient presents with difficulty in performing everyday tasks due continued shortness of breath. The GP prescribes a 6-month course of a fusion protein consisting of Staphylococcus aureus EDIN, Semliki Forest Virus fusogenic protein translocation domain and EGF TM in nebuliser form, 80 μg to be taken monthly. Following discussion with the physician, the patient selects the most appropriate nebuliser for her personal situation from a range of suitable devices. After a single dose of the EDIN-SFV fusogenic protein translocation domain-EGF fusion protein the patient experiences reduced sputum volume (to 10 ml) and an improvement in FEV1 (to 85%). Further treatment enhances these parameters further and improves quality of life.

### Example 43

A 38 year old female suffering from COPD presents at her GP. Treatment with bronchodilators has provided only minimal reversal of her impaired airway function. The GP prescribes a 6-month course of a fusion protein consisting of C. botulinum Exoenzyme C3, SER virus F protein translocation domain and EGF TM in nebuliser form, 80 μg to be taken monthly. Following discussion with the physician, the patient selects the most appropriate nebuliser for their personal situation from a range of suitable devices. After a single dose of the C3-F protein translocation domain-EGF fusion protein the patient experiences reduced expectoration improved maximal airway flow and lung emptying. Further treatment enhances these parameters further and improves quality of life.

### Example 44

A 54 year old male suffering from asthma presents at his GP. Despite treatment with standard asthma therapies, the patient obtains only partial relief from acute asthmatic episodes. The GP prescribes a 6-month course of a fusion protein consisting of Pokeweed antiviral protein, H_{N} and EGF TM in nebuliser form, 80 μg to be taken monthly. Following discussion with the physician, the patient selects the most appropriate nebuliser for his personal situation from a range of suitable devices. After a single dose of the Pokeweed antiviral protein-H_{N}-EGF fusion protein the patient experiences a decrease in the severity of acute asthmatic episodes. Further treatment enhances these parameters further and improves quality of life.

### Example 45

A 70 year old male suffering from chronic bronchitis (FEV1 reduced to 75% of normal predicted value; daily sputum volume of 25 ml) presents at his GP. Despite treatment with inhaled steroids, the patient presents with difficulty in performing everyday tasks due continued shortness of breath. The GP prescribes a 6-month course of a fusion protein consisting of Gelonin, VSV glycoprotein G and EGF TM in nebuliser form, 80 μg to be taken monthly. Following discussion with the physician, the patient selects the most appropriate nebuliser for her personal situation from a range of suitable devices. After a single dose of the Gelonin-VSV glycoprotein G-EGF fusion protein the patient experiences reduced sputum volume (to 10 ml) and an improvement in FEV1 (to 85%). Further treatment enhances these parameters further and improves quality of life.

### Example 46

A 68 year old male suffering from COPD presents at his GP. Treatment with bronchodilators has provided only minimal reversal of his impaired airway function. The GP prescribes a 6-month course of a fusion protein consisting of diphtheria toxin, Influenza virus haemagglutinin and EGF TM in nebuliser form, 80 μg to be taken monthly. Following discussion with the physician, the patient selects the most appropriate nebuliser for their personal situation from a range of suitable devices. After a single dose of the DT-influenza virus haemagglutinin-EGF fusion protein the patient experiences reduced expectoration improved maximal airway flow and lung emptying. Further treatment enhances these parameters further and improves quality of life.

### Example 47

A 63 year old female suffering from asthma presents at her GP. Despite treatment with standard asthma therapies, the patient obtains only partial relief from acute asthmatic episodes. The GP prescribes a 6-month course of a fusion protein consisting of Shiga toxin A-chain, SER virus F protein translocation domain and EGF TM in nebuliser form, 80 μg to be taken monthly. Following discussion with the physician, the patient selects the most appropriate nebuliser for his personal situation from a range of suitable devices. After a single dose of the Shiga toxin A-chain-SER virus F protein translocation domain-EGF fusion protein the patient experiences a decrease in the severity of acute asthmatic episodes. Further treatment enhances these parameters further and improves quality of life.

### Example 48

A 53 year old female suffering from chronic bronchitis (FEV1 reduced to 75% of normal predicted value; daily sputum volume of 25 ml) presents at her GP. Despite treatment with inhaled steroids, the patient presents with difficulty in performing everyday tasks due continued shortness of breath. The GP prescribes a 6-month course of a fusion protein consisting of Iris agglutinin r, foamy virus envelope glycoprotein translocation domain and EGF TM in nebuliser form, 80 μg to be taken monthly. Following discussion with the physician, the patient selects the most appropriate nebuliser for her personal situation from a range of suitable devices. After a single dose of the Iris agglutinin r-Foamy virus envelope glycoprotein translocation domain-EGF fusion protein the patient experiences reduced sputum volume (to 10 ml) and an improvement in FEV1 (to 85%). Further treatment enhances these parameters further and improves quality of life.

### Example 49

A 44 year old female suffering from COPD presents at her GP. Treatment with bronchodilators has provided only minimal reversal of her impaired airway function. The GP prescribes a 6-month course of a fusion protein consisting of Abrin A-chain, Semliki Forest Virus fusogenic protein translocation domain and EGF TM in nebuliser form, 80 μg to be taken monthly. Following discussion with the physician, the patient selects the most appropriate nebuliser for their personal situation from a range of suitable devices. After a single dose of the A-chain-SFV fusogenic protein translocation domain-EGF fusion protein the patient experiences reduced expectoration improved maximal airway flow and lung emptying. Further treatment enhances these parameters further and improves quality of life.

### Example 50

A 20 year old male suffering from asthma presents at his GP. Despite treatment with standard asthma therapies, the patient obtains only partial relief from acute asthmatic episodes. The GP prescribes a 6-month course of a fusion protein consisting of Shiga-like toxin A chain, diphtheria toxin translocation domain and EGF TM in nebuliser form, 80 μg to be taken monthly. Following discussion with the physician, the patient selects the most appropriate nebuliser for his personal situation from a range of suitable devices. After a single dose of the Shiga-like toxin A-chain-DT₁₉₄₋₃₈₀-EGF fusion protein the patient experiences a decrease in the severity of acute asthmatic episodes. Further treatment enhances these parameters further and improves quality of life.

### Example 51

A 65 year old male suffering from chronic bronchitis (FEV1 reduced to 75% of normal predicted value; daily sputum volume of 25 ml) presents at his GP. Despite treatment with inhaled steroids, the patient presents with difficulty in performing everyday tasks due continued shortness of breath. The GP prescribes a 6-month course of a fusion protein consisting of Cobra cytotoxin C1, VSV glycoprotein G and EGF TM in nebuliser form, 80 μg to be taken monthly. Following discussion with the physician, the patient selects the most appropriate nebuliser for her personal situation from a range of suitable devices. After a single dose of the C1-VSV glycoprotein G-EGF fusion protein the patient experiences reduced sputum volume (to 10 ml) and an improvement in FEV1 (to 85%). Further treatment enhances these parameters further and improves quality of life.

### Example 52

A 47 year old male suffering from COPD presents at his GP. Treatment with bronchodilators has provided only minimal reversal of his impaired airway function. The GP prescribes a 6-month course of a fusion protein consisting of Iris agglutinin b, H_{N} and EGF TM in nebuliser form, 80 μg to be taken monthly. Following discussion with the physician, the patient selects the most appropriate nebuliser for their personal situation from a range of suitable devices. After a single dose of the Iris agglutinin b-H_{N}-EGF fusion protein the patient experiences reduced expectoration improved maximal airway flow and lung emptying. Further treatment enhances these parameters further and improves quality of life.

### Example 53

A 41 year old male suffering from asthma presents at his GP. Despite treatment with standard asthma therapies, the patient obtains only partial relief from acute asthmatic episodes. The GP prescribes a 6-month course of a fusion protein consisting of Mistletoe lectin A subunit, SER virus F protein translocation domain and EGF TM in nebuliser form, 80 μg to be taken monthly. Following discussion with the physician, the patient selects the most appropriate nebuliser for his personal situation from a range of suitable devices. After a single dose of the lectin A subunit-F protein translocation domain-EGF fusion protein the patient experiences a decrease in the severity of acute asthmatic episodes. Further treatment enhances these parameters further and improves quality of life.

### Example 54

A 42 year old female suffering from chronic bronchitis (FEV1 reduced to 75% of normal predicted value; daily sputum volume of 25 ml) presents at her GP. Despite treatment with inhaled steroids, the patient presents with difficulty in performing everyday tasks due continued shortness of breath. The GP prescribes a 6-month course of a fusion protein consisting of Dianthin 30, domain II of Pseudomonas aeruginosa Exotoxin A (ETA) and EGF TM in nebuliser form, 80 μg to be taken monthly. Following discussion with the physician, the patient selects the most appropriate nebuliser for her personal situation from a range of suitable devices. After a single dose of the Dianthin 30-PE₄₀₅₋₆₁₃-EGF fusion protein the patient experiences reduced sputum volume (to 10 ml) and an improvement in FEV1 (to 85%). Further treatment enhances these parameters further and improves quality of life.

### Example 55

A 58 year old female suffering from COPD presents at her GP. Treatment with bronchodilators has provided only minimal reversal of her impaired airway function. The GP prescribes a 6-month course of a fusion protein consisting of Cobra Cytotoxin C2, Semliki Forest Virus fusogenic protein translocation domain and EGF TM in nebuliser form, 80 μg to be taken monthly. Following discussion with the physician, the patient selects the most appropriate nebuliser for their personal situation from a range of suitable devices. After a single dose of the C2-SFV fusogenic protein translocation domain-EGF fusion protein the patient experiences reduced expectoration improved maximal airway flow and lung emptying. Further treatment enhances these parameters further and improves quality of life.

### Example 56

A 17 year old female suffering from asthma presents at her GP. Despite treatment with standard asthma therapies, the patient obtains only partial relief from acute asthmatic episodes. The GP prescribes a 6-month course of a fusion protein consisting of Russells viper Cytotoxic phospholipase A2, Foamy virus envelope glycoprotein translocation domain and EGF TM in nebuliser form, 80 μg to be taken monthly. Following discussion with the physician, the patient selects the most appropriate nebuliser for his personal situation from a range of suitable devices. After a single dose of the A2-Foamy virus envelope glycoprotein translocation domain-EGF fusion protein the patient experiences a decrease in the severity of acute asthmatic episodes. Further treatment enhances these parameters further and improves quality of life.

### Example 57

A 51 year old male suffering from chronic bronchitis (FEV1 reduced to 75% of normal predicted value; daily sputum volume of 25 ml) presents at his GP. Despite treatment with inhaled steroids, the patient presents with difficulty in performing everyday tasks due continued shortness of breath. The GP prescribes a 6-month course of a fusion protein consisting of Cinnamomim type II ribosome inactivating protein, H_{N} and EGF TM in nebuliser form, 80 μg to be taken monthly. Following discussion with the physician, the patient selects the most appropriate nebuliser for her personal situation from a range of suitable devices. After a single dose of the Cinnamomim type II ribosome inactivating protein-H_{N}-EGF fusion protein the patient experiences reduced sputum volume (to 10 ml) and an improvement in FEV1 (to 85%). Further treatment enhances these parameters further and improves quality of life.

### Example 58

A 38 year old male suffering from COPD presents at his GP. Treatment with bronchodilators has provided only minimal reversal of his impaired airway function. The GP prescribes a 6-month course of a fusion protein consisting of IRIP type I ribosome inactivating protein, Influenza virus HA and EGF TM in nebuliser form, 80 μg to be taken monthly. Following discussion with the physician, the patient selects the most appropriate nebuliser for their personal situation from a range of suitable devices. After a single dose of the IRIP type I ribosome inactivating protein -HA-EGF fusion protein the patient experiences reduced expectoration improved maximal airway flow and lung emptying. Further treatment enhances these parameters further and improves quality of life.

### Example 59

A 35 year old male suffering from asthma presents at his GP. Despite treatment with standard asthma therapies, the patient obtains only partial relief from acute asthmatic episodes. The GP prescribes a 6-month course of a fusion protein consisting of WGA A-chain, diphtheria toxin translocation domain and EGF TM in nebuliser form, 80 μg to be taken monthly. Following discussion with the physician, the patient selects the most appropriate nebuliser for his personal situation from a range of suitable devices. After a single dose of the WGA A-chain-DT₁₉₄₋₃₈₀-EGF fusion protein the patient experiences a decrease in the severity of asthmatic episodes. Further treatment enhances these parameters further and improves quality of life.

### Example 60

A 59 year old female suffering from chronic bronchitis (FEV1 reduced to 75% of normal predicted value; daily sputum volume of 25 ml) presents at her GP. Despite treatment with inhaled steroids, the patient presents with difficulty in performing everyday tasks due continued shortness of breath. The GP prescribes a 6-month course of a fusion protein consisting of Trichosanthin, VSV glycoprotein G and EGF TM in nebuliser form, 80 μg to be taken monthly. Following discussion with the physician, the patient selects the most appropriate nebuliser for her personal situation from a range of suitable devices. After a single dose of the Trichosanthin-VSV glycoprotein G-EGF fusion protein the patient experiences reduced sputum volume (to 10 ml) and an improvement in (to 85%). Further treatment enhances these parameters further and improves quality of life.

### SEQ ID List

SEQ ID1 DNA sequence of LH_{N}/B
SEQ ID2 DNA sequence of LH_{N}/C
SEQ ID3 DNA sequence of the EGF linker
SEQ ID4 DNA sequence of the EGF-C fusion
SEQ ID5 Protein sequence of the EGF-C fusion
SEQ ID6 DNA sequence of the EGF-B fusion
SEQ ID7 Protein sequence of the EGF-B fusion
SEQ ID8 DNA sequence of the RGD linker
SEQ ID9 DNA sequence of the RGD-C fusion
SEQ ID10 Protein sequence of the RGD-C fusion
SEQ ID11 DNA sequence of the cyclic RGD linker
SEQ ID12 DNA sequence of the cyclic RGD-C fusion
SEQ ID13 Protein sequence of the cyclic RGD-C fusion
SEQ ID14 DNA sequence of the LC/C-RGD-H_{N}/C linker
SEQ ID15 DNA sequence of the LC/C-RGD-H_{N}/C fusion
SEQ ID16 Protein sequence of the LC/C-RGD-H_{N}/C fusion
SEQ ID17 DNA sequence of the fully synthesised LC/C-RGD-H_{N}/C fusion
SEQ ID18 Protein sequence of the fully synthesised LC/C-RGD-H_{N}/C fusion
SEQ ID19 DNA sequence of the fully synthesised EGF-LH_{N}/C fusion
SEQ ID20 Protein sequence of the fully synthesised EGF-LH_{N}/C fusion
SEQ ID21 Protein sequence from influenza virus
SEQ ID22 Substance P peptide sequence
SEQ ID23 Substance P peptide sequence with N-terminal Cys
SEQ ID24 Protein sequence for factor Xa
SEQ ID25 Protein sequence for enterokinase
SEQ ID26 Protein sequence for TEV protease
SEQ ID27 Protein sequence for precission
SEQ ID28 Protein sequence for thrombin
SEQ ID29 Integrin binding peptide sequence
SEQ ID30 Integrin binding peptide sequence
SEQ ID31 Cyclic RGD peptide
SEQ ID32 Linear integrin binding sequence
SEQ ID33 Cyclic integrin binding sequence

What is claimed is:

1. A polypeptide comprising:
(a) a cytotoxic toxin;
(b) a targeting domain that selectively binds to a target cell that is a mucus-secreting cell; and
(c) a translocating domain that translocates the cytotoxic toxin into the target cell.

2. A polypeptide according to claim 1, wherein said mucus-secreting cell is selected from the group consisting of consisting of epithelial goblet cells; submucosal gland mucus-secreting; Clara cells; serous cells; sensory efferent C-fibres; non-adrenergic non-cholinergic neural system fibres; and combinations thereof.

3. A polypeptide according to claim 1, wherein the mucus-secreting cell is a serous cell.

4. A polypeptide according to claim 1, wherein said targeting moiety is selected from the group consisting of epidermal growth factor (EGF), vascular endothelial growth factor, platelet-derived growth factor, keratinocyte growth factor, hepatocyte growth factor, transforming growth factor alpha, transforming growth factor beta.

5. A polypeptide according to claim 1, wherein the cytotoxic toxin is selected from the group consisting of bacterial cytotoxins, fungal cytotoxins, plant cytotoxins and animal cytotoxins.

6. A polypeptide according to claim 1, wherein the cytotoxin is selected from the group consisting of Helicobacter pylori VacA cytotoxin; Pseudomonas aeruginosa Exotoxin A (ETA); Bordatella pertussis Pertussis toxin (PT); Bordatella pertussis Adenylate cyclase toxin (CYA); E. coli Heat labile enterotoxin (LT); E. coli Shiga-like toxin A chain; Vibrio cholerae Cholera toxin; Staphylococcus aureus EDIN; Clostridial botulinum Exoenzyme C3; Corynebacterium diphtheriae Diphtheria toxin; Shigella dysenteriae Shiga-toxin A chain; Aspergillus giganteus α-sarcin; Naja oxiana Cytotoxin C1; Naja oxiana Cytotoxin C2; Russells viper Cytotoxic phospholipase A2; Iris hollandica IRIP type I ribosome inactivating protein; Iris hollandica Iris agglutinin b; Iris hollandica Iris agglutinin r; Cinnamomum camphora Cinnamomin type II ribosome inactivating protein; Ricinus communis Ricin A-chain; Abrus precatorius Abrin A-chain; Phytolacca Americana Pokeweed antiviral protein; Viscum album Mistletoe lectin A subunit; Saponaria officinalis Saporin 6; Triticum vulgaris Wheatgerm agglutinin A chain; Diathus caryophyllus Dianthin 30; Gelonium multiflorum Gelonin; and Tricosanthes kirilowii Trichosanthin.

7. A nucleic acid encoding a polypeptide according to claim 1.

8. A pharmaceutical composition for topical administration to a patient suffering from mucus hypersecretion, comprising:
(i) a polypeptide according to claim 1; and
(ii) a formulation component selected from the group consisting of an excipient, an adjuvant and a propellant.

9. A method of treating hypersecretion of mucus, comprising administering to a patient in need thereof, a therapeutically effective amount of a polypeptide according to claim 1; wherein following said administration, hypersecretion of mucus is reduced.

10. A method of treating chronic obstructive pulmonary disease (COPD), comprising administering to a patient in need thereof, a therapeutically effective amount of a polypeptide according to claim 1; wherein following said administration, hypersecretion of mucus is reduced.

11. A method of treating asthma, comprising administering to a patient in need thereof, a therapeutically effective amount of a polypeptide according to claim 1; wherein following said administration, hypersecretion of mucus is reduced.

12. A polypeptide according to claim 1, wherein the target cell is:
(i) a cell that secretes mucins and/or aqueous components of mucus; and/or
(ii) a neuronal cell that controls or directs mucus secretion.


SEQUENCE LISTING (33 sequences)

SEQ ID NO: 1 - DNA, 2631 bp, Unknown
  DNA sequence of LHN/B
ggatccatgccggttaccatcaacaacttcaactacaacgacccgatcgacaacaacaacatcattatgatggaaccgccgttcgcacgtggtaccggacgttactacaaggcttttaagatcaccgaccgtatctggatcatcccggaacgttacaccttcggttacaaacctgaggacttcaacaagagtagcgggattttcaatcgtgacgtctgcgagtactatgatccagattatctgaataccaacgataagaagaacatattccttcagactatgattaaactcttcaaccgtatcaaaagcaaaccgctcggtgaaaaactcctcgaaatgattatcaacggtatcccgtacctcggtgaccgtcgtgtcccgcttgaagagttcaacaccaacatcgcaagcgtcaccgtcaacaaactcatcagcaacccaggtgaagtcgaacgtaaaaaaggtatcttcgcaaacctcatcatcttcggtccgggtccggtcctcaacgaaaacgaaaccatcgacatcggtatccagaaccacttcgcaagccgtgaaggtttcggtggtatcatgcagatgaaattctgcccggaatacgtcagtgtcttcaacaacgtccaggaaaacaaaggtgcaagcatcttcaaccgtcgtggttacttcagcgacccggcactcatcctcatgcatgaactcatccacgtcctccacggtctctacggtatcaaagttgacgacctcccgatcgtcccgaacgagaagaaattcttcatgcagagcaccgacgcaatccaggctgaggaactctacaccttcggtggccaagacccaagtatcataaccccgtccaccgacaaaagcatctacgacaaagtcctccagaacttcaggggtatcgtggacagactcaacaaagtcctcgtctgcatcagcgacccgaacatcaatatcaacatatacaagaacaagttcaaagacaagtacaaattcgtcgaggacagcgaaggcaaatacagcatcgacgtagaaagtttcgacaagctctacaaaagcctcatgttcggtttcaccgaaaccaacatcgccgagaactacaagatcaagacaagggcaagttacttcagcgacagcctcccgcctgtcaaaatcaagaacctcttagacaacgagatttacacaattgaagagggcttcaacatcagtgacaaagacatggagaaggaatacagaggtcagaacaaggctatcaacaaacaggcatacgaggagatcagcaaagaacacctcgcagtctacaagatccagatgtgcgtcgacgaagaaaagctgtacgacgacgacgacaaagaccgttggggttcttcgctgcagtgcatcgacgttgacaacgaagacctgttcttcatcgctgacaaaaacagcttcagtgacgacctgagcaaaaacgaacgtatcgaatacaacacccagagcaactacatcgaaaacgacttcccgatcaacgaactgatcctggacaccgacctgataagtaaaatcgaactgccgagcgaaaacaccgaaagtctgaccgacttcaacgttgacgttccggtttacgaaaaacagccggctatcaagaaaatcttcaccgacgaaaacaccatcttccagtacctgtacagccagaccttcccgctggacatccgtgacatcagtctgaccagcagtttcgacgacgctctgctgttcagcaacaaagtttacagtttcttcagcatggactacatcaaaaccgctaacaaagttgttgaagcagggctgttcgctggttgggttaaacagatcgttaacgacttcgttatcgaagctaacaaaagcaacactatggacaaaatcgctgacatcagtctgatcgttccgtacatcggtctggctctgaacgttggtaacgaaaccgctaaaggtaactttgaaaacgctttcgagatcgctggtgcaagcatcctgctggagttcatcccggaactgctgatcccggttgttggtgctttcctgctggaaagttacatcgacaacaaaaacaagatcatcaaaaccatcgacaacgctctgaccaaacgtaacgaaaaatggagtgatatgtacggtctgatcgttgctcagtggctgagcaccgtcaacacccagttctacaccatcaaagaaggtatgtacaaagctctgaactaccaggctcaggctctggaagagatcatcaaataccgttacaacatctacagtgagaaggaaaagagtaacatcaacatcgacttcaacgacatcaacagcaaactgaacgaaggtatcaaccaggctatcgacaacatcaacaacttcatcaacggttgcagtgttagctacctgatgaagaagatgatcccgctggctgttgaaaaactgctggacttcgacaacaccctgaaaaagaacctgctgaactacatcgacgaaaacaagctgtacctgatcggtagtgctgaatacgaaaaaagtaaagtgaacaaatacctgaagaccatcatgccgttcgacctgagtatctacaccaacgacaccatcctgatcgaaatgttcaacaaatacaactctctagactagaagctt

SEQ ID NO: 2 - DNA, 2620 bp, Unknown
  DNA sequence of LHN/C
ggatccatgccgatcaccatcaacaacttcaactacagcgatccggtggataacaaaaacatcctgtacctggatacccatctgaataccctggcgaacgaaccggaaaaagcgtttcgtatcaccggcaacatttgggttattccggatcgttttagccgtaacagcaacccgaatctgaataaaccgccgcgtgttaccagcccgaaaagcggttattacgatccgaactatctgagcaccgatagcgataaagataccttcctgaaagaaatcatcaaactgttcaaacgcatcaacagccgtgaaattggcgaagaactgatctatcgcctgagcaccgatattccgtttccgggcaacaacaacaccccgatcaacacctttgatttcgatgtggatttcaacagcgttgatgttaaaacccgccagggtaacaattgggtgaaaaccggcagcattaacccaagcgtgattattaccggtccgcgcgaaaacattattgatccggaaaccagcacctttaaactgaccaacaacacctttgcggcgcaggaaggttttggcgcgctgagcattattagcattagcccgcgctttatgctgacctatagcaacgcgaccaacgatgttggtgaaggccgtttcagcaaaagcgaattttgcatggacccgatcctgatcctgatgcatgaactgaaccatgcgatgcataacctgtatggcatcgcgattccgaacgatcagaccattagcagcgtgaccagcaacatcttttacagccagtacaacgtgaaactggaatatgcggaaatctatgcgtttggcggtccgaccattgatctgattccgaaaagcgcgcgcaaatacttcgaagaaaaagcgctggattactatcgcagcattgcgaaacgtctgaacagcattaccaccgcgaatccgagcagcttcaacaaatatatcggcgaatataaacagaaactgatccgcaaatatcgctttgtggtggaaagcagcggcgaagttaccgttaaccgcaataaattcgtggaactgtacaacgaactgacccagatcttcaccgaatttaactatgcgaaaatctataacgtgcagaaccgtaaaatctacctgagcaacgtgtataccccggtgaccgcgaatattctggatgataacgtgtacgatatccagaacggctttaacatcccgaaaagcaacctgaacgttctgtttatgggccagaacctgagccgtaatccggcgctgcgtaaagtgaacccggaaaacatgctgtacctgttcaccaaattttgcgtcgacgcgattgatggtcgtagcctgtacaacaaaaccctgcagtgtcgtgaactgctggtgaaaaacaccgatctgccgtttattggcgatatcagcgatgtgaaaaccgatatcttcctgcgcaaagatatcaacgaagaaaccgaagtgatctactacccggataacgtgagcgttgatcaggtgatcctgagcaaaaacaccagcgaacatggtcagctggatctgctgtatccgagcattgatagcgaaagcgaaattctgccgggcgaaaaccaggtgttttacgataaccgtacccagaacgtggattacctgaacagctattactacctggaaagccagaaactgagcgataacgtggaagattttacctttacccgcagcattgaagaagcgctggataacagcgcgaaagtttacacctattttccgaccctggcgaacaaagttaatgcgggtgttcagggcggtctgtttctgatgtgggcgaacgatgtggtggaagatttcaccaccaacatcctgcgtaaagataccctggataaaatcagcgatgttagcgcgattattccgtatattggtccggcgctgaacattagcaatagcgtgcgtcgtggcaattttaccgaagcgtttgcggttaccggtgtgaccattctgctggaagcgtttccggaatttaccattccggcgctgggtgcgtttgtgatctatagcaaagtgcaggaacgcaacgaaatcatcaaaaccatcgataactgcctggaacagcgtattaaacgctggaaagatagctatgaatggatgatgggcacctggctgagccgtattatcacccagttcaacaacatcagctaccagatgtacgatagcctgaactatcaggcgggtgcgattaaagcgaaaatcgatctggaatacaaaaaatacagcggcagcgataaagaaaacatcaaaagccaggttgaaaacctgaaaaacagcctggatgtgaaaattagcgaagcgatgaataacatcaacaaattcatccgcgaatgcagcgtgacctacctgttcaaaaacatgctgccgaaagtgatcgatgaactgaacgaatttgatcgcaacaccaaagcgaaactgatcaacctgatcgatagccacaacattattctggtgggcgaagtggataaactgaaagcgaaagttaacaacagcttccagaacaccatcccgtttaacatcttcagctataccaacaacagcctgctgaaagatatcatcaacgaatacttcaatctagactaataagctt

SEQ ID NO: 3 - DNA, 249 bp, Unknown
  DNA sequence of EGF linker
ggatccgtcgacctgcagggtctagaaggcggtggcggtagcggcggtggcggtagcggcggtggcggtagcgcactagacaactctgactctgaatgcccgctgtctcacgacggttactgcctgcacgacggtgtttgcatgtacatcgaagctctggacaaatacgcttgcaactgcgttgttggttacatcggtgaacgttgccagtaccgtgacctgaaatggtgggaactgcgttgaaagctt

SEQ ID NO: 4 - DNA, 2838 bp, Unknown
  DNA sequence of EGF-C fusion
ggatccgaattcatgccgatcaccatcaacaacttcaactacagcgatccggtggataacaaaaacatcctgtacctggatacccatctgaataccctggcgaacgaaccggaaaaagcgtttcgtatcaccggcaacatttgggttattccggatcgttttagccgtaacagcaacccgaatctgaataaaccgccgcgtgttaccagcccgaaaagcggttattacgatccgaactatctgagcaccgatagcgataaagataccttcctgaaagaaatcatcaaactgttcaaacgcatcaacagccgtgaaattggcgaagaactgatctatcgcctgagcaccgatattccgtttccgggcaacaacaacaccccgatcaacacctttgatttcgatgtggatttcaacagcgttgatgttaaaacccgccagggtaacaattgggtgaaaaccggcagcattaacccgagcgtgattattaccggtccgcgcgaaaacattattgatccggaaaccagcacctttaaactgaccaacaacacctttgcggcgcaggaaggttttggcgcgctgagcattattagcattagcccgcgctttatgctgacctatagcaacgcgaccaacgatgttggtgaaggccgtttcagcaaaagcgaattttgcatggacccgatcctgatcctgatgcatgaactgaaccatgcgatgcataacctgtatggcatcgcgattccgaacgatcagaccattagcagcgtgaccagcaacatcttttacagccagtacaacgtgaaactggaatatgcggaaatctatgcgtttggcggtccgaccattgatctgattccgaaaagcgcgcgcaaatacttcgaagaaaaagcgctggattactatcgcagcattgcgaaacgtctgaacagcattaccaccgcgaatccgagcagcttcaacaaatatatcggcgaatataaacagaaactgatccgcaaatatcgctttgtggtggaaagcagcggcgaagttaccgttaaccgcaataaattcgtggaactgtacaacgaactgacccagatcttcaccgaatttaactatgcgaaaatctataacgtgcagaaccgtaaaatctacctgagcaacgtgtataccccggtgaccgcgaatattctggatgataacgtgtacgatatccagaacggctttaacatcccgaaaagcaacctgaacgttctgtttatgggccagaacctgagccgtaatccggcgctgcgtaaagtgaacccggaaaacatgctgtacctgttcaccaaattttgcgtcgacgcgattgatggtcgtagcctgtacaacaaaaccctgcagtgtcgtgaactgctggtgaaaaacaccgatctgccgtttattggcgatatcagcgatgtgaaaaccgatatcttcctgcgcaaagatatcaacgaagaaaccgaagtgatctactacccggataacgtgagcgttgatcaggtgatcctgagcaaaaacaccagcgaacatggtcagctggatctgctgtatccgagcattgatagcgaaagcgaaattctgccgggcgaaaaccaggtgttttacgataaccgtacccagaacgtggattacctgaacagctattactacctggaaagccagaaactgagcgataacgtggaagattttacctttacccgcagcattgaagaagcgctggataacagcgcgaaagtttacacctattttccgaccctggcgaacaaagttaatgcgggtgttcagggcggtctgtttctgatgtgggcgaacgatgtggtggaagatttcaccaccaacatcctgcgtaaagataccctggataaaatcagcgatgttagcgcgattattccgtatattggtccggcgctgaacattagcaatagcgtgcgtcgtggcaattttaccgaagcgtttgcggttaccggtgtgaccattctgctggaagcgtttccggaatttaccattccggcgctgggtgcgtttgtgatctatagcaaagtgcaggaacgcaacgaaatcatcaaaaccatcgataactgcctggaacagcgtattaaacgctggaaagatagctatgaatggatgatgggcacctggctgagccgtattatcacccagttcaacaacatcagctaccagatgtacgatagcctgaactatcaggcgggtgcgattaaagcgaaaatcgatctggaatacaaaaaatacagcggcagcgataaagaaaacatcaaaagccaggttgaaaacctgaaaaacagcctggatgtgaaaattagcgaagcgatgaataacatcaacaaattcatccgcgaatgcagcgtgacctacctgttcaaaaacatgctgccgaaagtgatcgatgaactgaacgaatttgatcgcaacaccaaagcgaaactgatcaacctgatcgatagccacaacattattctggtgggcgaagtggataaactgaaagcgaaagttaacaacagcttccagaacaccatcccgtttaacatcttcagctataccaacaacagcctgctgaaagatatcatcaacgaatacttcaatctagaaggtggcggtgggtccggtggcggtggctcaggcgggggcggtagcgcactagacaactctgactctgaatgcccgctgtctcacgacggttactgcctgcacgacggtgtttgcatgtacatcgaagctctggacaaatacgcttgcaactgcgttgttggttacatcggtgaacgttgccagtaccgtgacctgaaatggtgggaactgcgttgaaagctt

SEQ ID NO: 5 - protein, 945 aa, Unknown
  Protein sequence of the EGF-C fusion
GSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRSLYNKTLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEGGGGSGGGGSGGGGSALDNSDSECPLSHDGYCLHDGVCMYIEALDKYACNCVVGYIGERCQYRDLKWWELRKL

SEQ ID NO: 6 - DNA, 2850 bp, Unknown
  DNA sequence of the EGF-B fusion
ggatccatgccggttaccatcaacaacttcaactacaacgacccgatcgacaacaacaacatcattatgatggaaccgccgttcgcacgtggtaccggacgttactacaaggcttttaagatcaccgaccgtatctggatcatcccggaacgttacaccttcggttacaaacctgaggacttcaacaagagtagcgggattttcaatcgtgacgtctgcgagtactatgatccagattatctgaataccaacgataagaagaacatattccttcagactatgattaaactcttcaaccgtatcaaaagcaaaccgctcggtgaaaaactcctcgaaatgattatcaacggtatcccgtacctcggtgaccgtcgtgtcccgcttgaagagttcaacaccaacatcgcaagcgtcaccgtcaacaaactcatcagcaacccaggtgaagtcgaacgtaaaaaaggtatcttcgcaaacctcatcatcttcggtccgggtccggtcctcaacgaaaacgaaaccatcgacatcggtatccagaaccacttcgcaagccgtgaaggtttcggtggtatcatgcagatgaaattctgcccggaatacgtcagtgtcttcaacaacgtccaggaaaacaaaggtgcaagcatcttcaaccgtcgtggttacttcagcgacccggcactcatcctcatgcatgaactcatccacgtcctccacggtctctacggtatcaaagttgacgacctcccgatcgtcccgaacgagaagaaattcttcatgcagagcaccgacgcaatccaggctgaggaactctacaccttcggtggccaagacccaagtatcataaccccgtccaccgacaaaagcatctacgacaaagtcctccagaacttcaggggtatcgtggacagactcaacaaagtcctcgtctgcatcagcgacccgaacatcaatatcaacatatacaagaacaagttcaaagacaagtacaaattcgtcgaggacagcgaaggcaaatacagcatcgacgtagaaagtttcgacaagctctacaaaagcctcatgttcggtttcaccgaaaccaacatcgccgagaactacaagatcaagacaagggcaagttacttcagcgacagcctcccgcctgtcaaaatcaagaacctcttagacaacgagatttacacaattgaagagggcttcaacatcagtgacaaagacatggagaaggaatacagaggtcagaacaaggctatcaacaaacaggcatacgaggagatcagcaaagaacacctcgcagtctacaagatccagatgtgcgtcgacgaagaaaagctgtacgacgacgacgacaaagaccgttggggttcttcgctgcagtgcatcgacgttgacaacgaagacctgttcttcatcgctgacaaaaacagcttcagtgacgacctgagcaaaaacgaacgtatcgaatacaacacccagagcaactacatcgaaaacgacttcccgatcaacgaactgatcctggacaccgacctgataagtaaaatcgaactgccgagcgaaaacaccgaaagtctgaccgacttcaacgttgacgttccggtttacgaaaaacagccggctatcaagaaaatcttcaccgacgaaaacaccatcttccagtacctgtacagccagaccttcccgctggacatccgtgacatcagtctgaccagcagtttcgacgacgctctgctgttcagcaacaaagtttacagtttcttcagcatggactacatcaaaaccgctaacaaagttgttgaagcagggctgttcgctggttgggttaaacagatcgttaacgacttcgttatcgaagctaacaaaagcaacactatggacaaaatcgctgacatcagtctgatcgttccgtacatcggtctggctctgaacgttggtaacgaaaccgctaaaggtaactttgaaaacgctttcgagatcgctggtgcaagcatcctgctggagttcatcccggaactgctgatcccggttgttggtgctttcctgctggaaagttacatcgacaacaaaaacaagatcatcaaaaccatcgacaacgctctgaccaaacgtaacgaaaaatggagtgatatgtacggtctgatcgttgctcagtggctgagcaccgtcaacacccagttctacaccatcaaagaaggtatgtacaaagctctgaactaccaggctcaggctctggaagagatcatcaaataccgttacaacatctacagtgagaaggaaaagagtaacatcaacatcgacttcaacgacatcaacagcaaactgaacgaaggtatcaaccaggctatcgacaacatcaacaacttcatcaacggttgcagtgttagctacctgatgaagaagatgatcccgctggctgttgaaaaactgctggacttcgacaacaccctgaaaaagaacctgctgaactacatcgacgaaaacaagctgtacctgatcggtagtgctgaatacgaaaaaagtaaagtgaacaaatacctgaagaccatcatgccgttcgacctgagtatctacaccaacgacaccatcctgatcgaaatgttcaacaaatacaactctctagatctagaaggtggcggtgggtccggtggcggtggctcaggcgggggcggtagcgcactagacaactctgactctgaatgcccgctgtctcacgacggttactgcctgcacgacggtgtttgcatgtacatcgaagctctggacaaatacgcttgcaactgcgttgttggttacatcggtgaacgttgccagtaccgtgacctgaaatggtgggaactgcgttgaaagctt

SEQ ID NO: 7 - protein, 949 aa, Unknown
  Protein sequence of the EGF-B fusion
GSMPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCVDEEKLYDDDDKDRWGSSLQCIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSLDLEGGGGSGGGGSGGGGSALDNSDSECPLSHDGYCLHDGVCMYIEALDKYACNCVVGYIGERCQYRDLKWWELRKL

SEQ ID NO: 8 - DNA, 120 bp, Unknown
  DNA sequence of the RGD linker
ggatccgtcgacctgcagggtctagaaggcggtggcggtagcggcggtggcggtagcggcggtggcggtagcgcactagtgggtggtcgtggtgacatgttcggtgcttgataaaagctt

SEQ ID NO: 9 - DNA, 2709 bp, Unknown
  DNA sequence of the RGD-C fusion
ggatccgaattcatgccgatcaccatcaacaacttcaactacagcgatccggtggataacaaaaacatcctgtacctggatacccatctgaataccctggcgaacgaaccggaaaaagcgtttcgtatcaccggcaacatttgggttattccggatcgttttagccgtaacagcaacccgaatctgaataaaccgccgcgtgttaccagcccgaaaagcggttattacgatccgaactatctgagcaccgatagcgataaagataccttcctgaaagaaatcatcaaactgttcaaacgcatcaacagccgtgaaattggcgaagaactgatctatcgcctgagcaccgatattccgtttccgggcaacaacaacaccccgatcaacacctttgatttcgatgtggatttcaacagcgttgatgttaaaacccgccagggtaacaattgggtgaaaaccggcagcattaacccgagcgtgattattaccggtccgcgcgaaaacattattgatccggaaaccagcacctttaaactgaccaacaacacctttgcggcgcaggaaggttttggcgcgctgagcattattagcattagcccgcgctttatgctgacctatagcaacgcgaccaacgatgttggtgaaggccgtttcagcaaaagcgaattttgcatggacccgatcctgatcctgatgcatgaactgaaccatgcgatgcataacctgtatggcatcgcgattccgaacgatcagaccattagcagcgtgaccagcaacatcttttacagccagtacaacgtgaaactggaatatgcggaaatctatgcgtttggcggtccgaccattgatctgattccgaaaagcgcgcgcaaatacttcgaagaaaaagcgctggattactatcgcagcattgcgaaacgtctgaacagcattaccaccgcgaatccgagcagcttcaacaaatatatcggcgaatataaacagaaactgatccgcaaatatcgctttgtggtggaaagcagcggcgaagttaccgttaaccgcaataaattcgtggaactgtacaacgaactgacccagatcttcaccgaatttaactatgcgaaaatctataacgtgcagaaccgtaaaatctacctgagcaacgtgtataccccggtgaccgcgaatattctggatgataacgtgtacgatatccagaacggctttaacatcccgaaaagcaacctgaacgttctgtttatgggccagaacctgagccgtaatccggcgctgcgtaaagtgaacccggaaaacatgctgtacctgttcaccaaattttgcgtcgacgcgattgatggtcgtagcctgtacaacaaaaccctgcagtgtcgtgaactgctggtgaaaaacaccgatctgccgtttattggcgatatcagcgatgtgaaaaccgatatcttcctgcgcaaagatatcaacgaagaaaccgaagtgatctactacccggataacgtgagcgttgatcaggtgatcctgagcaaaaacaccagcgaacatggtcagctggatctgctgtatccgagcattgatagcgaaagcgaaattctgccgggcgaaaaccaggtgttttacgataaccgtacccagaacgtggattacctgaacagctattactacctggaaagccagaaactgagcgataacgtggaagattttacctttacccgcagcattgaagaagcgctggataacagcgcgaaagtttacacctattttccgaccctggcgaacaaagttaatgcgggtgttcagggcggtctgtttctgatgtgggcgaacgatgtggtggaagatttcaccaccaacatcctgcgtaaagataccctggataaaatcagcgatgttagcgcgattattccgtatattggtccggcgctgaacattagcaatagcgtgcgtcgtggcaattttaccgaagcgtttgcggttaccggtgtgaccattctgctggaagcgtttccggaatttaccattccggcgctgggtgcgtttgtgatctatagcaaagtgcaggaacgcaacgaaatcatcaaaaccatcgataactgcctggaacagcgtattaaacgctggaaagatagctatgaatggatgatgggcacctggctgagccgtattatcacccagttcaacaacatcagctaccagatgtacgatagcctgaactatcaggcgggtgcgattaaagcgaaaatcgatctggaatacaaaaaatacagcggcagcgataaagaaaacatcaaaagccaggttgaaaacctgaaaaacagcctggatgtgaaaattagcgaagcgatgaataacatcaacaaattcatccgcgaatgcagcgtgacctacctgttcaaaaacatgctgccgaaagtgatcgatgaactgaacgaatttgatcgcaacaccaaagcgaaactgatcaacctgatcgatagccacaacattattctggtgggcgaagtggataaactgaaagcgaaagttaacaacagcttccagaacaccatcccgtttaacatcttcagctataccaacaacagcctgctgaaagatatcatcaacgaatacttcaatctagaaggcggtggcggtagcggcggtggcggtagcggcggtggcggtagcgcactagtgggtggtcgtggtgacatgttcggtgcttgataaaagctt

SEQ ID NO: 10 - protein, 901 aa, Unknown
  Protein sequence of RGD-C fusion
GSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRSLYNKTLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEGGGGSGGGGSGGGGSALVGGRGDMFGAKL

SEQ ID NO: 11 - DNA, 126 bp, Unknown
  DNA sequence of cyclic RGD linker
ggatccgtcgacctgcagggtctagaaggcggtggcggtagcggcggtggcggtagcggcggtggcggtagcgcactagtgggtggttgccgtggtgacatgttcggttgcgcttgataaaagctt

SEQ ID NO: 12 - DNA, 2715 bp, Unknown
  DNA sequence of cyclic RGD-C fusion
ggatccgaattcatgccgatcaccatcaacaacttcaactacagcgatccggtggataacaaaaacatcctgtacctggatacccatctgaataccctggcgaacgaaccggaaaaagcgtttcgtatcaccggcaacatttgggttattccggatcgttttagccgtaacagcaacccgaatctgaataaaccgccgcgtgttaccagcccgaaaagcggttattacgatccgaactatctgagcaccgatagcgataaagataccttcctgaaagaaatcatcaaactgttcaaacgcatcaacagccgtgaaattggcgaagaactgatctatcgcctgagcaccgatattccgtttccgggcaacaacaacaccccgatcaacacctttgatttcgatgtggatttcaacagcgttgatgttaaaacccgccagggtaacaattgggtgaaaaccggcagcattaacccgagcgtgattattaccggtccgcgcgaaaacattattgatccggaaaccagcacctttaaactgaccaacaacacctttgcggcgcaggaaggttttggcgcgctgagcattattagcattagcccgcgctttatgctgacctatagcaacgcgaccaacgatgttggtgaaggccgtttcagcaaaagcgaattttgcatggacccgatcctgatcctgatgcatgaactgaaccatgcgatgcataacctgtatggcatcgcgattccgaacgatcagaccattagcagcgtgaccagcaacatcttttacagccagtacaacgtgaaactggaatatgcggaaatctatgcgtttggcggtccgaccattgatctgattccgaaaagcgcgcgcaaatacttcgaagaaaaagcgctggattactatcgcagcattgcgaaacgtctgaacagcattaccaccgcgaatccgagcagcttcaacaaatatatcggcgaatataaacagaaactgatccgcaaatatcgctttgtggtggaaagcagcggcgaagttaccgttaaccgcaataaattcgtggaactgtacaacgaactgacccagatcttcaccgaatttaactatgcgaaaatctataacgtgcagaaccgtaaaatctacctgagcaacgtgtataccccggtgaccgcgaatattctggatgataacgtgtacgatatccagaacggctttaacatcccgaaaagcaacctgaacgttctgtttatgggccagaacctgagccgtaatccggcgctgcgtaaagtgaacccggaaaacatgctgtacctgttcaccaaattttgcgtcgacgcgattgatggtcgtagcctgtacaacaaaaccctgcagtgtcgtgaactgctggtgaaaaacaccgatctgccgtttattggcgatatcagcgatgtgaaaaccgatatcttcctgcgcaaagatatcaacgaagaaaccgaagtgatctactacccggataacgtgagcgttgatcaggtgatcctgagcaaaaacaccagcgaacatggtcagctggatctgctgtatccgagcattgatagcgaaagcgaaattctgccgggcgaaaaccaggtgttttacgataaccgtacccagaacgtggattacctgaacagctattactacctggaaagccagaaactgagcgataacgtggaagattttacctttacccgcagcattgaagaagcgctggataacagcgcgaaagtttacacctattttccgaccctggcgaacaaagttaatgcgggtgttcagggcggtctgtttctgatgtgggcgaacgatgtggtggaagatttcaccaccaacatcctgcgtaaagataccctggataaaatcagcgatgttagcgcgattattccgtatattggtccggcgctgaacattagcaatagcgtgcgtcgtggcaattttaccgaagcgtttgcggttaccggtgtgaccattctgctggaagcgtttccggaatttaccattccggcgctgggtgcgtttgtgatctatagcaaagtgcaggaacgcaacgaaatcatcaaaaccatcgataactgcctggaacagcgtattaaacgctggaaagatagctatgaatggatgatgggcacctggctgagccgtattatcacccagttcaacaacatcagctaccagatgtacgatagcctgaactatcaggcgggtgcgattaaagcgaaaatcgatctggaatacaaaaaatacagcggcagcgataaagaaaacatcaaaagccaggttgaaaacctgaaaaacagcctggatgtgaaaattagcgaagcgatgaataacatcaacaaattcatccgcgaatgcagcgtgacctacctgttcaaaaacatgctgccgaaagtgatcgatgaactgaacgaatttgatcgcaacaccaaagcgaaactgatcaacctgatcgatagccacaacattattctggtgggcgaagtggataaactgaaagcgaaagttaacaacagcttccagaacaccatcccgtttaacatcttcagctataccaacaacagcctgctgaaagatatcatcaacgaatacttcaatctagaaggcggtggcggtagcggcggtggcggtagcggcggtggcggtagcgcactagtgggtggttgccgtggtgacatgttcggttgcgcttgataaaagctt

SEQ ID NO: 13 - protein, 903 aa, Unknown
  Protein sequence of cyclic RGD-C fusion
GSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRSLYNKTLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEGGGGSGGGGSGGGGSALVGGCRGDMFGCAKL

SEQ ID NO: 14 - DNA, 156 bp, Unknown
  DNA sequence of the LC/C-RGD-HN/C LINKER
ggatccacgcacgtcgacgcgattgatggtcgtggtggtcgtggtgacatgttcggtgctgcgctagcgggcggtggcggtagcggcggtggcggtagcggcggtggcggtagcgcactagtgctgcagacgcacggtctagaatgataaaagctt

SEQ ID NO: 15 - DNA, 2733 bp, Unknown
  DNA sequence of the LC/C-RGD-HN/C fusion
ggatccgaattcatgccgatcaccatcaacaacttcaactacagcgatccggtggataacaaaaacatcctgtacctggatacccatctgaataccctggcgaacgaaccggaaaaagcgtttcgtatcaccggcaacatttgggttattccggatcgttttagccgtaacagcaacccgaatctgaataaaccgccgcgtgttaccagcccgaaaagcggttattacgatccgaactatctgagcaccgatagcgataaagataccttcctgaaagaaatcatcaaactgttcaaacgcatcaacagccgtgaaattggcgaagaactgatctatcgcctgagcaccgatattccgtttccgggcaacaacaacaccccgatcaacacctttgatttcgatgtggatttcaacagcgttgatgttaaaacccgccagggtaacaattgggtgaaaaccggcagcattaacccgagcgtgattattaccggtccgcgcgaaaacattattgatccggaaaccagcacctttaaactgaccaacaacacctttgcggcgcaggaaggttttggcgcgctgagcattattagcattagcccgcgctttatgctgacctatagcaacgcgaccaacgatgttggtgaaggccgtttcagcaaaagcgaattttgcatggacccgatcctgatcctgatgcatgaactgaaccatgcgatgcataacctgtatggcatcgcgattccgaacgatcagaccattagcagcgtgaccagcaacatcttttacagccagtacaacgtgaaactggaatatgcggaaatctatgcgtttggcggtccgaccattgatctgattccgaaaagcgcgcgcaaatacttcgaagaaaaagcgctggattactatcgcagcattgcgaaacgtctgaacagcattaccaccgcgaatccgagcagcttcaacaaatatatcggcgaatataaacagaaactgatccgcaaatatcgctttgtggtggaaagcagcggcgaagttaccgttaaccgcaataaattcgtggaactgtacaacgaactgacccagatcttcaccgaatttaactatgcgaaaatctataacgtgcagaaccgtaaaatctacctgagcaacgtgtataccccggtgaccgcgaatattctggatgataacgtgtacgatatccagaacggctttaacatcccgaaaagcaacctgaacgttctgtttatgggccagaacctgagccgtaatccggcgctgcgtaaagtgaacccggaaaacatgctgtacctgttcaccaaattttgcgtcgacgcgattgatggtcgtggtggtcgtggtgacatgttcggtgctgcgctagcgggcggtggcggtagcggcggtggcggtagcggcggtggcggtagcgcactagtgctgcagtgtcgtgaactgctggtgaaaaacaccgatctgccgtttattggcgatatcagcgatgtgaaaaccgatatcttcctgcgcaaagatatcaacgaagaaaccgaagtgatctactacccggataacgtgagcgttgatcaggtgatcctgagcaaaaacaccagcgaacatggtcagctggatctgctgtatccgagcattgatagcgaaagcgaaattctgccgggcgaaaaccaggtgttttacgataaccgtacccagaacgtggattacctgaacagctattactacctggaaagccagaaactgagcgataacgtggaagattttacctttacccgcagcattgaagaagcgctggataacagcgcgaaagtttacacctattttccgaccctggcgaacaaagttaatgcgggtgttcagggcggtctgtttctgatgtgggcgaacgatgtggtggaagatttcaccaccaacatcctgcgtaaagataccctggataaaatcagcgatgttagcgcgattattccgtatattggtccggcgctgaacattagcaatagcgtgcgtcgtggcaattttaccgaagcgtttgcggttaccggtgtgaccattctgctggaagcgtttccggaatttaccattccggcgctgggtgcgtttgtgatctatagcaaagtgcaggaacgcaacgaaatcatcaaaaccatcgataactgcctggaacagcgtattaaacgctggaaagatagctatgaatggatgatgggcacctggctgagccgtattatcacccagttcaacaacatcagctaccagatgtacgatagcctgaactatcaggcgggtgcgattaaagcgaaaatcgatctggaatacaaaaaatacagcggcagcgataaagaaaacatcaaaagccaggttgaaaacctgaaaaacagcctggatgtgaaaattagcgaagcgatgaataacatcaacaaattcatccgcgaatgcagcgtgacctacctgttcaaaaacatgctgccgaaagtgatcgatgaactgaacgaatttgatcgcaacaccaaagcgaaactgatcaacctgatcgatagccacaacattattctggtgggcgaagtggataaactgaaagcgaaagttaacaacagcttccagaacaccatcccgtttaacatcttcagctataccaacaacagcctgctgaaagatatcatcaacgaatacttcaatctagaagcactagcgagtgggcaccatcaccatcaccattaatgaaagctt

SEQ ID NO: 16 - protein, 909 aa, Unknown
  Protein sequence of the LC/C-RGD-HN/C fusion
GSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRGGRGDMFGAALAGGGGSGGGGSGGGGSALVLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEALASGHHHHHHKL

SEQ ID NO: 17 - DNA, 2721 bp, Unknown
  DNA sequence of the fully synthesised LC/C-RGD-HN/C fusion
catatgggctccgaatttatgccgataacaattaacaatttcaattactcggatccggtggacaacaaaaacattctgtatctggatacacatttaaatactcttgcgaatgaaccagaaaaagcgttcagaattacgggaaatatctgggtcatcccggatcgcttttcgagaaactcaaaccccaacctgaacaaaccgccccgtgttacaagtccgaaaagcggctattacgatccaaactacctttcgaccgactcggacaaagatacgtttcttaaagagataattaaactgtttaaacgtatcaattcacgcgaaattggggaagagttaatttaccgcctctccaccgacattccgtttccaggcaataacaatacaccgattaacacctttgatttcgacgtggacttcaacagcgtggatgttaaaacgcgccagggtaataactgggtaaagacgggatcgattaacccgagtgttattatcaccggtcctcgcgaaaatatcatagacccggaaactagcacgtttaaacttactaataacacattcgcggcccaagaagggttcggcgccctgtcaattataagcatcagtccgcgctttatgctgacttacagtaatgctactaatgacgtgggtgagggccggttctctaaatcagaattttgcatggatccaatcctgattctgatgcatgagctgaatcacgctatgcacaatctgtatggtattgctattccgaacgatcagacaattagttcagtgacgtctaacatattctattctcaatataatgtgaaattggagtatgcggaaatttatgcatttggtggcccaaccatcgatcttatcccaaaatccgcgcgcaagtatttcgaagagaaagcattagattattaccggtctatcgcaaagcgtctgaatagcataactacggctaatccgagttcgtttaacaaatatattggcgaatataaacagaaactgatccgtaaatatcgtttcgtagtggaatcatccggtgaagttacagtcaatcgtaataaatttgtggagttatacaatgagctgacccaaatcttcaccgaattcaactatgctaaaatttataatgttcagaaccgcaaaatctacctgagtaacgtgtatacgcctgtaacagccaatattctggatgacaacgtgtatgatatccagaatggctttaacatacctaaaagtaacttgaatgttctctttatgggtcaaaatctttcccgcaatccggctctccgaaaggtaaatccggaaaacatgctctatcttttcaccaaattttgcgtcgacgcaatcgatggacgtggtgggagaggtgatatgtttggggccgcattagcgggtggcgggggatccggcggtggcggtagtggcgggggcggaagcgcgctggtactgcagtgtcgcgaacttttagttaagaatactgatctgccattcattggtgatatctcagatgtcaagaccgatattttcctccgtaaagatatcaatgaggaaacagaggtaatttactatccggataatgtatctgtcgatcaggtcattctgtccaaaaatacctctgaacacggtcaactggatctgctctacccctcgattgactccgaatctgaaatcctccctggagaaaaccaggtcttttatgacaatcgtacccagaacgtggactacttaaactcttattactatttggagagccaaaagttgtccgataacgttgaagactttacttttacccgatctatagaagaggcattagacaactcggcgaaggtttacacctatttccctaccttagccaataaagtgaacgcaggtgtgcagggagggctgtttttgatgtgggccaatgatgtcgttgaggatttcacaaccaacattctgcgcaaagacactttagataaaatctcagatgtatcggcgatcattccctacattggccctgcccttaacatttctaattccgttcgtcgcggcaattttactgaggcgtttgctgtcaccggtgtgacgatcttgctggaggcttttcctgaatttaccattcccgcactgggggcattcgttatctacagtaaggttcaggaacggaacgaaattataaaaacaatcgataattgcctggaacagcgtatcaaacggtggaaagatagctacgaatggatgatgggcacgtggttgagccgcataattacgcagtttaataacatctcatatcaaatgtatgactccctgaattaccaggcgggcgcgattaaagccaaaatcgatctggagtacaaaaagtattcaggcagcgacaaagagaacattaaaagtcaggttgaaaacctgaagaattcactggatgtgaaaatcagcgaagccatgaataacattaataaattcatccgtgaatgtagtgtgacctatctctttaagaatatgttgccgaaagttatcgatgagctgaacgagtttgatcgaaataccaaagcaaagctgattaatttaattgacagccataatattatactggtcggcgaagtggataaactgaaggccaaggtaaacaattcttttcaaaacacgataccattcaacatcttttcttatacgaataacagccttctgaaggacattattaacgaatattttaatttggaagccttggctagcggataatgaaagctt

SEQ ID NO: 18 - protein, 902 aa, Unknown
  Protein sequence of the fully synthesised LC/C-RGD-HN/C fusion
MGSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRGGRGDMFGAALAGGGGSGGGGSGGGGSALVLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEALASG

SEQ ID NO: 19 - DNA, 2859 bp, Unknown
  DNA sequence of the fully synthesised EGF-LHN/C fusion
catatgatttccgaatttggctcggagttcatgccaattacgattaacaattttaactatagtgatccggtggataataaaaacattttatacctggatacccacttgaatactcttgccaatgagcctgaaaaagcctttcgcataacgggtaacatttgggtcattccggaccgttttagccggaactctaaccctaatctgaataaacctccgcgtgtcacgtctccgaaaagtgggtattacgatccaaattatctgagtaccgattcagacaaggatacgtttctgaaagaaatcataaaacttttcaaaagaatcaactcccgtgaaatcggtgaagagctgatctaccgtctgtcgacggacattccttttccgggaaacaataacactcccattaataccttcgactttgatgtcgatttcaactcagtcgatgtgaaaacccgccagggtaataactgggttaaaactggatccattaacccgtccgttattatcacaggtcctcgtgaaaatattatagatcctgagacctccacgttcaagctgacgaataacacttttgcggcacaggaagggtttggtgccctttcaattatctctatctctccgcgcttcatgttaacgtattctaacgcaaccaacgatgttggcgagggccgcttcagcaaaagtgaattctgtatggatcccattctgatcttgatgcatgagcttaaccacgctatgcataatctttatggtattgcaatcccaaacgatcagacgatctccagcgttacatctaacatattctacagccaatataatgtgaagctcgaatatgcagagatttacgccttcggtgggccgaccattgacctcattccaaagtctgcccgtaagtactttgaggaaaaagcgttggattactatcgtagcatcgcgaaacgcctgaattcaattacaactgcaaacccatctagcttcaacaaatacatcggagaatataaacaaaagctgatacgcaaatatcgctttgtggtcgaatcgtccggggaagtgacagttaatcgaaataaatttgttgaactctataatgaattaacgcagatcttcacagaatttaattatgctaaaatctataatgtacagaaccggaaaatttatctcagtaatgtatacacaccggtgactgctaacattctggacgataacgtctacgatattcaaaatggctttaatatcccgaagagcaacttgaatgtcctcttcatggggcagaacttgtcacgtaacccagcgctgcgaaaagttaacccagaaaatatgttgtacctctttacaaaattctgtgtagacgccattgacggacgctcactgtacaacaaaaccctgcaatgccgtgaacttctggttaagaacaccgacctgccgttcattggggacatcagtgatgtcaaaacggatatttttcttcggaaggatattaatgaggaaaccgaagtgatatactatcctgacaatgtgtcggtagatcaggtaatcctgagtaagaacaccagcgagcatgggcagctggatctgttgtatccgagcattgacagcgagtcggaaatactgcccggcgaaaatcaagttttttatgacaatcggacccagaatgttgattatctgaatagttactattacttggagagccaaaaattatcagataatgtggaagactttacctttacccggtctatcgaagaggcgctggataacagcgcgaaagtttacacttattttcccacgctcgcaaacaaagttaatgctggcgtacagggtggattatttcttatgtgggcgaatgatgtggtagaggactttacaaccaacatcctgcgcaaagacactttagacaaaatttctgacgtctcggccattatcccgtatataggtccggccttaaacataagcaattcggttcgccgtggcaacttcacagaagccttcgctgtgactggtgtgaccattctgttggaagcatttcctgagtttacgatcccggctctgggcgcatttgtaatttactctaaagttcaggaacgaaatgaaattataaaaactatcgataattgcctggaacagcgtatcaagagatggaaggattcctatgagtggatgatggggacctggctgtcaagaattatcacacagtttaataacatatcctatcaaatgtatgatagcttaaactatcaagcaggagcgataaaggcgaaaattgacctggaatacaagaaatattctggttcggataaagagaatattaaaagtcaggtggaaaatctgaaaaatagtttagatgtcaaaatttctgaggcgatgaataacattaacaaattcatccgcgagtgcagtgtaacttatttgtttaagaatatgttacccaaagttatcgacgaactgaatgaatttgatcgtaataccaaagccaaattgatcaacctcatcgactctcataacatcattctggtgggagaagtcgacaaactgaaagctaaggtgaataacagcttccagaatacaattccgtttaatattttctcatacaccaataactcgctgcttaaagatattatcaacgaatattttaatctggagggtggcggtggcagtggcggtggcggatccggcggtggcggtagcgcactggataattcagattccgaatgtccactgtcacacgatggttattgtcttcatgatggcgtgtgcatgtatatagaagcgttagataaatacgcttgcaactgcgtggttggctatatcggcgaacgttgtcagtatcgtgatttaaagtggtgggaattacgctaatgaaagctt

SEQ ID NO: 20 - protein, 948 aa, Unknown
  Protein sequence of the fully synthesised EGF-LHN/C fusion
MISEFGSEFMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVDAIDGRSLYNKTLQCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNLEGGGGSGGGGSGGGGSALDNSDSECPLSHDGYCLHDGVCMYIEALDKYACNCVVGYIGERCQYRDLKWWELR

SEQ ID NO: 21 - protein, 23 aa, Influenza virus
GLFGAIAGFIENGWEGMIDGWYG

SEQ ID NO: 22 - protein, 12 aa, Unknown
  Substance P peptide sequence
RPKPQQFFGLMC

SEQ ID NO: 23 - protein, 12 aa, Unknown
  Substance P peptide sequence with N-terminal Cys
CRPKPQQFFGLM

SEQ ID NO: 24 - protein, 4 aa, Unknown
  Factor Xa
IEGR

SEQ ID NO: 25 - protein, 5 aa, Unknown
  Enterokinase
DDDDK

SEQ ID NO: 26 - protein, 8 aa, Unknown
  TEV protease
EXXYXQSG

SEQ ID NO: 27 - protein, 8 aa, unknown
  Precission
LEVLFQGP

SEQ ID NO: 28 - protein, 6 aa, unknown
  Thrombin
LVPRGS

SEQ ID NO: 29 - protein, 9 aa, Unknown
  Integrin binding peptide sequence
GGRGDMFGA

SEQ ID NO: 30 - protein, 11 aa, Unknown
  Integrin binding peptide sequence
GGCRGDMFGCA

SEQ ID NO: 31 - protein, 5 aa, Unknown
  cyclic RGD peptide
RGDFV

SEQ ID NO: 32 - protein, 10 aa, Unknown
  linear integrin binding sequence
PLAEIDGIEL

SEQ ID NO: 33 - protein, 12 aa, Unknown
  cyclic integrin binding sequence
CPLAEIDGIELC